(19) United States
(12) Patent Application Publication
(10) Pub. No.: US 2023/0242638 A1
(43) Pub. Date: Aug. 3, 2023

(54) CHIMERIC ANTIGEN RECEPTOR TARGETING CLDN18.2 AND USE THEREOF

(71) Applicant: SHANGHAI JIAO TONG UNIVERSITY, Shanghai (CN)

(72) Inventors: Xuanming YANG, Shanghai (CN); Huihui ZHANG, Shanghai (CN); Xiaoqing ZHANG, Shanghai (CN); Fanlin LI, Shanghai (CN); Jie LIANG, Shanghai (CN)

(73) Assignee: SHANGHAI LONGYAO BIOTECHNOLOGY INC., LTD, Shanghai (CN)

(21) Appl. No.: 18/000,674

(22) PCT Filed: Jun. 4, 2021
(86) PCT No.: PCT/CN2021/098259

§ 371 Date: Dec. 2, 2022

(30) Foreign Application Priority Data
Jun. 5, 2020 (CN) 202010507858.2

(51) Int. Cl. C07K 16/28; C12N 15/63; A61P 35/00; C07K 14/705

(52) U.S. Cl. CPC C07K 16/28; C12N 15/63; A61P 35/00; C07K 14/70575; C07K 2319/03; C07K 2319/02; C07K 2317/622

(57) ABSTRACT

A fusion protein including a chimeric antigen receptor (CAR) targeting claudin 18.2 (CLDN18.2), and a synergistic domain that can improve a tumor cells killing capacity of said chimeric antigen receptor targeting CLDN18.2. A method for treating a tumor, including administering a cell that expresses the fusion protein to a subject in need thereof.

### TECHNICAL FIELD

The present application relates to the field of biomedicine and specifically to a chimeric antigen receptor targeting CLDN18.2 and use thereof.

### BACKGROUND OF THE INVENTION

In adoptive cell therapy, for example, chimeric antigen receptor T cells (CAR-T cells) are artificially modified tumor killer cells that combine the targeted recognition function of antibodies with the tumor killing function of T cells, rendering them a major breakthrough in the field of tumor immunotherapy. However, the therapeutic effect of CAR-T in the treatment of solid tumors such as gastric cancer and pancreatic cancer is not yet ideal; thus, the expected discovery of novel CAR-T structures and new targets acts as the key to the treatment of solid tumors with CAR-T.

CLN18 is a member of the Claudins protein family, in which CLDN18.1 and CLDN18.2 are alternative splicing variants of CLDN 18. In normal tissues, CLDN18.1 is mainly expressed in the lung, while CLDN18.2 is only expressed in gastric mucosal epithelial cells. However, CLDN18.2 is expressed in a variety of tumor tissues, such as gastric cancer, pancreatic cancer, esophageal cancer, ovarian cancer, lung cancer etc., making it an ideal target for tumor therapeutic CAR-T. Despite the fact that huge success has been achieved in the treatment of hematological tumors using existing CAR structures, yet highly unsatisfactory efficacy has been observed in the treatment of solid tumors using exactly the same CAR structures mainly due to the immunosuppressive microenvironment in these solid tumors. Therefore, it is highly imperative to develop new targets and new CAR structures for the treatment of cancers like pancreatic cancer and gastric cancer.

### SUMMARY OF THE INVENTION

To address the disadvantage that the structure of the existing chimeric antigen receptor targeting CLDN18.2 delivers unsatisfactory and poor killing capacity to CLDN18.2-positive tumor cells, the present application provides a chimeric antigen receptor targeting CLDN18.2 and use thereof, including a fusion protein, a nucleic acid molecule, a vector and a cell with high specific activity against Claudin18.2, as well as preparation method, pharmaceutical composition and use thereof; also provided in the present application are methods of improving the tumor cell killing capacity and T cell proliferation capacity of the chimeric antigen receptor targeting CLDN18.2.

The present application provides a fusion protein, comprising a) a chimeric antigen receptor (CAR) targeting CLDN18.2; and b) a synergistic domain, and the synergistic domain enables the improvement in tumor cell killing capacity of the chimeric antigen receptor (CAR) targeting CLDN18.2.

In some embodiments, the synergistic domain comprises a costimulatory synergistic domain and the costimulatory synergistic domain comprises a protein or functional fragment thereof selected from the following group: OX40 and OX40L.

In some embodiments, the costimulatory synergistic domain comprises an amino acid sequence as shown in any one of SEQ ID NOs: 23-24.

In some embodiments, the synergistic domain comprises a chemotactically synergistic domain and the chemotactically synergistic domain comprises a protein or functional fragment thereof selected from the following group: CCR7 and CXCR5.

In some embodiments, the chemotactically synergistic domain comprises an amino acid sequence as shown in any one of SEQ ID NOs: 25-26.

In some embodiments, a C-terminus of the chimeric antigen receptor targeting CLDN18.2 is directly or indirectly linked to an N-terminus of the synergistic domain.

In some embodiments, the linkage is achieved via a linker.

In some embodiments, the linker comprises an amino acid sequence as shown in SEQ ID NO: 27.

In some embodiments, the fusion protein consists of a single-chain structure.

In some embodiments, the fusion protein comprises the chimeric antigen receptor targeting CLDN18.2, the linker and the synergistic domain in sequence from the N-terminus to the C-terminus.

In some embodiments, the chimeric antigen receptor targeting CLDN18.2 comprises a CLDN18.2-binding domain, a transmembrane domain, a costimulatory domain and an intracellular signaling domain, wherein the CLDN18.2-binding domain comprises an antibody or fragment thereof that specifically binds to CLDN18.2.

In some embodiments, the antibody is a single-chain antibody.

In some embodiments, the antibody comprises an amino acid sequence as shown in any one of SEQ ID NOs: 28-29.

In some embodiments, the transmembrane domain comprises a transmembrane domain derived from a protein selected from the group: alpha, beta, or zeta chain of a T cell receptor, CD28, CD3e, CD45, CD4, CD5, CD8a, CD9, CD16, CD22, CD33, CD37, CD64, CD80, CD86, CD134, CD137, and CD154.

In some embodiments, the transmembrane domain comprises an amino acid sequence as shown in SEQ ID NO: 31.

In some embodiments, the costimulatory domain comprises a costimulatory domain derived from a protein selected from the group: CD28, 4-1BB, OX40, and ICOS.

In some embodiments, the costimulatory domain comprises an amino acid sequence as shown in SEQ ID NO: 32.

In some embodiments, the intracellular signaling domain comprises a signaling domain derived from CD3ζ.

In some embodiments, the intracellular signaling domain comprises an amino acid sequence as shown in SEQ ID NO: 33.

In some embodiments, the chimeric antigen receptor targeting CLDN18.2 comprises an amino acid sequence as shown in any one of SEQ ID NOs: 23-33.

In another aspect, the present application provides one or more isolated nucleic acid molecules that encode the fusion protein or fragment thereof.

In another aspect, the present application provides a vector that comprises the nucleic acid.

In another aspect, the present application provides a cell that comprises the vector and/or the fusion protein.

In another aspect, the present application provides a method for preparing the fusion protein, which includes the following step: synthesizing the fusion protein, and/or culturing the cells under conditions required for expressing the fusion protein.

In another aspect, the present application provides a pharmaceutical composition, which comprises the fusion protein and optionally a pharmaceutically acceptable adjuvant.

In another aspect, the present application provides a use of the fusion protein and/or the pharmaceutical composition in the preparation of a medicament for the treatment of a tumor.

In another aspect, the present application provides a use of the fusion protein and/or the pharmaceutical composition in the preparation of a medicament, wherein the tumor includes lymphoma and/or pancreatic cancer.

In another aspect, the present application provides a method for treating a tumor, which includes administering the fusion protein and/or the pharmaceutical composition to a subject in need thereof in an amount effective for the treatment of a cancer.

In another aspect, the present application provides a method for administering the fusion protein and/or the pharmaceutical composition for treating a tumor, wherein the tumor includes lymphoma and/or pancreatic cancer.

In another aspect, the present application provides the fusion protein and/or the pharmaceutical composition, which are used for the treatment of a tumor.

In another aspect, the present application provides the fusion protein and/or the pharmaceutical composition, which are used for the treatment of a tumor, and the tumor includes lymphoma and/or pancreatic cancer.

In another aspect, the present application provides a method for improving the tumor cell killing capacity of a chimeric antigen receptor targeting CLDN18.2, which includes the following step: linking the chimeric antigen receptor targeting CLDN18.2 to a synergistic domain, wherein the synergistic domain comprises a protein or functional fragment thereof selected from the following groups: OX40, OX40L, CCR7, and CXCR5.

In some embodiments, the C-terminus of the chimeric antigen receptor targeting CLDN18.2 is directly or indirectly linked to the N-terminus of the synergistic domain.

In some embodiments, the linkage is achieved via a linker.

In some embodiments, the linker comprises an amino acid sequence as shown in SEQ ID NO: 27.

In some embodiments, the chimeric antigen receptor targeting CLDN18.2 is a chimeric antigen receptor targeting CLDN18.2 of the fusion protein.

In some embodiments, the synergistic domain comprises an amino acid sequence as shown in any one of SEQ ID NOs: 23-26.

In another respect, the present application provides a method for improving the proliferation capacity of T cells comprising a chimeric antigen receptor targeting CLDN18.2, which includes the following step: linking the chimeric antigen receptor targeting CLDN18.2 to a synergistic domain, wherein the synergistic domain comprises a protein or functional fragment thereof selected from the following groups: OX40, OX40L, CCR7, and CXCR5.

In some embodiments, the C-terminus of the chimeric antigen receptor targeting CLDN18.2 is directly or indirectly linked to the N-terminus of the synergistic domain.

In some embodiments, the linkage is achieved via a linker.

In some embodiments, the linker comprises an amino acid sequence as shown in SEQ ID NO: 27.

In some embodiments, the chimeric antigen receptor targeting CLDN18.2 is a chimeric antigen receptor targeting CLDN18.2 of the fusion protein.

In some embodiments, the synergistic domain comprises an amino acid sequence as shown in any one of SEQ ID NOs: 23-26.

In some embodiments, the T cells are derived from PBMC.

The chimeric antigen receptor targeting CLDN18.2 provided in the present application delivers the advantageous effect of improving the activation capacity, proliferation capacity and/or tumor cell killing capacity of CAR-T by introducing a new costimulatory synergistic domain or a chemotactically synergistic domain independent of CAR.

Those skilled in the art can easily perceive other aspects and advantages of the present application from the detailed description below. In the following detailed description, only exemplary embodiments of the present application are shown and described. As those skilled in the art will recognize, the content of the present application enables those skilled in the art to make changes to the disclosed specific embodiments without departing from the spirit and scope of the invention involved in the present application. Correspondingly, the drawings and descriptions in the specification of the present application are merely exemplary, rather than restrictive.

### BRIEF DESCRIPTION OF THE DRAWING

The specific features of the invention to which this application relates are shown in the appended claims. The features and advantages of the invention to which this application relates will be better understood by reference to the exemplary embodiments and the accompanying drawings described in detail below. A brief description of the drawings is as follows:

FIG. 1 shows the structural diagram of the chimeric antigen receptor (CAR) targeting CLDN18.2 of the present application.

FIGS. 2A-2B show the titer determination results of the non-synergistic anti-CLDN18.2 CAR-T virus (A is virus Ab10BBZ, B is virus Ab362BBZ) of the present application.

FIGS. 3A-3D show the titer determination results of the costimulatory synergistic anti-CLDN18.2CAR-T virus (A is virus Ab10BBZ-OX40, B is virus Ab10BBZ-OX40L, C is virus Ab362BBZ-OX40 and D is virus Ab362BBZ-OX40L) of the present application.

FIGS. 4A-4B show the titer determination results of the chemotactically synergistic anti-CLDN18.2 CAR-T virus (A is virus Ab10BBZ-CXCR5 and B is virus Ab10BBZ-CCR7) of the present application.

FIGS. 5A-5H show the assay results of the expression of anti-CLDN18.2 CAR-T cells (A is Ab10BBZ CAR-T cell, B is Ab10BBZ-OX40 CAR-T cell, C is Ab362BBZ CAR-T cell, D is Ab362BBZ-OX40 CAR-T cell, E is Ab10BBZ-OX40L CAR-T cell, F is Ab362BBZ-OX40L CAR-T cell, G is Ab10BBZ-CCR7 CAR-T cell, and H is Ab10BBZ-CXCR5 CAR-T cell).

FIG. 6 shows the results of in vitro proliferation comparative experiment of anti-CLDN18.2 CAR-T cell (Ab362BBZ CAR-T cell, Ab362BBZ-OX40 CAR-T cell, Ab10BBZ CAR-T cell, Ab10BBZ-OX40 CAR-T cell, Ab10BBZ-CCR7 CAR-T cell, Ab10BBZ-CXCR5 CAR-T cell, Ab10BBZ-OX40L CAR-T cell and Ab362BBZ-OX40L CAR-T cell) of the present application. Statistical significance (P) is indicated by an asterisk: ** means P<0.01, and * means P<0.05.

FIG. 7 shows the detection results of the killing of CLDN18.2-positive tumor cells by the anti-CLDN18.2 CAR-T cells (Ab10BBZ CAR-T cell, Ab10BBZ-OX40 CAR-T cell and Ab10BBZ-OX40L CAR-T cell) of the present application. Statistical significance (P) is indicated by an asterisk: *** means P<0.001.

FIG. 8 shows the detection results of the killing of CLDN18.2-positive tumor cells by the anti-CLDN18.2 CAR-T cells (Ab10BBZ-CCR7 CAR-T cell, Ab10BBZ-CXCR7 CAR-T cell and Ab10BBZ-CXCR5 CAR-T cell) of the present application. Statistical significance (P) is indicated by an asterisk: *** means P<0.001, and * means P<0.05.

FIG. 9 shows the in vivo anti-tumor experimental results of the anti-CLDN18.2 CAR-T cells (Ab10BBZ CAR-T cell and Ab10BBZ-OX40 CAR-T cell) of the present application. Statistical significance (P) is indicated by an asterisk: ** means P<0.01.

FIG. 10 shows the in vivo anti-tumor experimental results of anti-CLDN18.2 CAR-T cells (Ab10BBZ CAR-T cell and Ab10BBZ-CXCR5 CAR-T cell) in this application. Statistical significance (P) is indicated by an asterisk: *** means P<0.001, and * means P<0.05.

### DETAILED DESCRIPTION

The implementation of the present application will be illustrated in the following specific examples, and other advantages and effects of the present application will be easily known by those familiar with this technology from the content disclosed in the specification.

### Definition of Terms

In the present application, the term “intracellular signaling domain” refers to the intracellular part of the molecule. The intracellular signal domain transduces effector functional signals and directs cells to perform specialized functions. Although an entire intracellular signaling domain may be used, in many cases, the use of an entire chain is not necessary. In the case of using a truncated portion of an intracellular signaling domain, such a truncated portion may be used in place of the entire chain as long as it transduces an effector functional signal. The term “intracellular signaling domain” is therefore intended to include any truncated portion of an intracellular signaling domain sufficient to transduce effector functional signals, for example, the CD3ζ.

In the present application, the term “single chain” refers to a molecule comprising amino acid monomers that are linearly linked via a peptide bond.

In the present application, the term “costimulatory domain” refers to an intracellular portion of a costimulatory molecule, or a truncated form thereof, which is capable of transducing a costimulatory signal (also referred to as a second signal), for example, CD28, 4-1BB, OX-40, and ICOS.

In the present application, the term “antibody or fragment thereof” includes immunologically binding agents extending to all antibodies from all species, including dimer, trimer and multimer antibodies, bispecific antibody, chimeric antibodies, human and humanized antibody, recombinant and modified antibodies and fragments thereof. The term “antibody or fragment thereof” may refer to any antibody-like molecule having an antigen binding region, and the term includes fragments of small-molecule substances such as Fab′, Fab, F(ab′)2, single-domain antibodies (DABs), Fv, scFv (single-chain Fv), linear antibodies, double antibodies etc. Techniques for preparing and using various antibody-based constructs and fragments are well known in the art.

In the present application, the term “transmembrane domain” refers to a portion of a CAR that extends across a cell membrane and anchors the CAR to the cell membrane. For example, the alpha, beta, or zeta chains of T cell receptors, CD28, CD3e, CD45, CD4, CD5, CD8a, CD9, CD16, CD22, CD33, CD37, CD64, CD80, CD86, CD134, CD137, and CD154.

In the present application, the term “linker” refers to a peptide having an amino acid sequence, which may be of synthetic origin. In the fusion protein according to the present invention, the linker is used to fuse the synergistic domain to the C- or N-terminus of the chimeric antigen receptor.

In the present application, the term “killing capacity” refers to killing cells by exposing the cells with an effective amount of an antibody, immunoconjugate, bispecific/multispecific molecule or composition. The method may include killing cells expressing CLDN18.2, optionally in the presence of effector cells, such as by CDC, apoptosis, ADCC, phagocytosis, or by a combination of two or more of these mechanisms. Cell expressing CLDN18.2 that can be killed using the fusion protein of the present invention include cancer cell such as neoplastic gastric, pancreatic, esophageal, pulmonary, ovarian, colonal, hepatic, head and neck, and cholecystic cells.

In the present application, the terms “specific binding”, “specific binding affinity” or “specific targeting” describe a molecule that binds to another molecule with a binding affinity that is higher than background binding. A binding domain (or a CAR comprising a binding domain or a fusion protein comprising a binding domain) is “specifically bound” to a target molecule if its binding or association with the target molecule has, for example, an affinity or Ka (i. e., the equilibrium association constant for a particular binding interaction, in 1/M) greater than or equal to about 10⁵M⁻¹.

In the present application, the term “directly or indirectly linked” refers to the linkage directly via a peptide bond, or indirect linkage via a linker or via a non-peptide connection.

In the present application, the terms “CLDN18.2-binding domain”, “extracellular antigen binding domain”, “extracellular domain” or “extracellular ligand-binding domain” refer to a portion of a CAR located outside a cell membrane and capable of binding to an antigen, target or ligand.

In the present application, the term “PBMC” or “human peripheral blood mononuclear cell” generally refers to cells having single nucleus in peripheral blood, for example, any blood cell (i.e., lymphocyte, monocyte or macrophage) having a round nucleus. These blood cells constitute the key components of the immune system in resisting infection and adapting to intruders. The lymphocyte population consists of CD4⁺ and CD8⁺ T cells, B cells and natural killer cells, CD14⁺ monocytes and basophils/neutrophils/eosinophils/dendritic cells. Typically, these cells are isolated from whole blood using FICOLL™, a hydrophilic polysaccharide that allows the blood to delaminate, in which monocytes and lymphocytes form a buffy coat below the plasma layer. For example, “PBMC” refers to a population of cells comprising at least T cells, and optionally NK cells and antigen presenting cells.

In the present application, the term “T cell” refers to thymus-derived cells that are involved in various cell-mediated immune responses, including thymocytes, naïve T lymphocytes, immature T lymphocytes, mature T lymphocytes, resting T lymphocytes, or activated T lymphocytes. Exemplary T cell populations may include, but are not limited to, helper T cells (HTL; CD4⁺ T cell), cytotoxic T cells (CTL; CD8⁺ T cell), CD4⁺CD8⁺ T cells, CD4⁻CD8⁻ T cells, or any other subset of T cells. Other exemplary T cell populations may include, but are not limited to, T cells expressing one or more of the following markers: CD3, CD4, CD8, CD27, CD28, CD45RA, CD45RO, CD62L, CD127, CD197, and HLA-DR, and may be further isolated using positive or negative selection techniques, if desired.

In the present application, the term “proliferation” refers to an increase in cell division (symmetrical or asymmetrical division of cells). “Proliferation” may refer to symmetrical or asymmetrical division of T cells. “Increased proliferation” occurs when an increase in the number of cells is observed in the treated sample compared to cells in the untreated sample.

In the present application, the term “subject” includes any human or non-human animal. The term “non-human animal” includes all vertebrates, such as mammals and non-mammals, for example, non-human primates, sheep, dogs, cats, cattle, horses, chickens, amphibians, and reptiles, and may be mammals, such as non-human primates, sheep, dogs, cats, cattle, and horses.

In the present application, the term “therapeutically effective dosage” refers to the amount of an antibody of the present application sufficient to prevent or alleviate symptoms associated with a disease or disorder (e.g., cancer). A therapeutically effective dosage is associated with the disease to be treated, wherein the actual effective dosage can be readily determined by those skilled in the art.

In the present application, the term “medicament” generally refers to a chemical compound or composition that, when properly administered to a patient, induces a desired therapeutic effect.

In the present application, the term “pharmaceutical composition” means a mixture of a drug containing one or more of the compounds described herein or physiologically/pharmaceutically acceptable salts or prodrug thereof with other chemical components, as well as other components such as physiologically/pharmaceutically acceptable vectors and excipients. The pharmaceutical composition is designed to aid in the administration to organisms and facilitate the absorption of the active ingredient to exert its respective biological activity. Therapeutic compositions should generally be sterile and stable under their corresponding manufacturing and storage conditions. The compositions may be formulated as solutions, micro emulsions, dispersants, liposomes or other ordered structures compatible with high antibody concentrations. Sterile injectable solutions may be prepared by incorporating the active compound (i. e., antibody or antibody moiety) in the desired amount along with one of the ingredients or combinations of ingredients listed above in a suitable solvent, followed by sterile filtration as required.

In the present application, the term “vector” generally refers to a nucleic acid molecule capable of transporting another nucleic acid to which it is attached. One type of vector is a “plasmid,” which refers to a circular double-stranded DNA ring into which other DNA segments can be ligated. Another type of vector is a viral vector in which other DNA segment can be ligated into the viral genome. Certain vectors are capable of performing autonomous replication in the host cell into which they are introduced (e.g., bacterial vectors having a bacterial origin of replication and episomal mammalian vectors). Other vectors (e.g., non-episomal mammalian vectors) may be incorporated into the genome of the host cell upon introduction into the host cell so as to replicate together with the host genome, such as naked RNA polynucleotides incapable of autonomous replication, naked DNA polynucleotides, polynucleotides consisting of DNA and RNA in the same strand, poly-lysine-coupled DNA or RNA, peptide-coupled DNA or RNA, liposome-coupled DNA etc. In addition, certain vectors are capable of directing the expression of genes operably linked to them. Such vectors are referred to herein as “recombinant expression vector” (or simply “expression vectors”). In general, expression vectors used in recombinant DNA techniques are generally in the form of plasmids. In the present specification, “plasmid” and “vector” are used interchangeably since plasmids are the most commonly used form of vector.

In the present application, the term “adjuvant” generally refers to any substance that aids or modulates the action of a medicament, including but not limited to immunological adjuvants that enhance or diversify the immune response to antigens.

In the present application, the term “tumor” or “tumor cell” generally refers to or describes a physiological condition in mammals typically characterized by unregulated cell growth. Examples of tumors include, but are not limited to, carcinoma, lymphoma, blastoma (including medulloblastoma and retinoblastoma), sarcoma (including liposarcoma and synoviosarcoma), neuroendocrine tumor (including carcinoid tumor, gastrinoma and islet cell carcinoma), mesothelioma, schwannoma (including acoustic neuroma), meningioma, adenocarcinoma and melanoma. The “tumor cell” further includes a “solid tumor”, which refers to a tumor selected from the following group: gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine cancer, salivary adenocarcinoma, renal cancer, prostate cancer, vulvar cancer, thyroid cancer, hepatic carcinoma, anal cancer, penile cancer, testicular cancer, esophageal cancer, biliary tumors, and head and neck cancer, which may be lymphoma and/or pancreatic cancer.

In the present application, the terms “CLDN18.2”, “CLD18.2”, “Claudin18.2”, “claudin18.2” or “claudin 18.2” include Claudin18, type 2. The term includes variants, homologs, orthologs and parallel homologs.

In the present application, the term “CLDN18.2-positive tumor” refers to a tumor cell expressing the CLDN18.2 protein on its surface. For the purpose of determining whether a cell expresses CLDN18.2 protein on the surface, it is considered that mRNA expression of CLDN18.2 is related to CLDN18.2 protein expression on the cell surface, and the expression of CLDN18.2 mRNA can be determined by a method selected from the group consisting of in situ hybridization and RT-PCR including quantitative RT-PCR. Alternatively, for example, the expression of CLDN18.2 protein on the cell surface can be determined by such methods as immunohistochemistry, FACS etc. using antibodies against CLDN18.2 protein. For example, a CLDN18.2-positive tumor could be a mammalian implant or a tumor obtained by subcutaneously inoculating CFPAC-1 tumor cells to B-NDG mice.

In the present application, the term “CLDN18.2-positive tumor cell” refers to a cell expressing the CLDN18.2 protein on its surface. For the purpose of determining whether a cell expresses CLDN18.2 protein on the surface, it is considered that the expression of CLDN18.2 mRNA is related to CLDN18.2 protein expression on the cell surface, and the expression of CLDN18.2 mRNA can be determined by a method selected from the group consisting of in situ hybridization and RT-PCR including quantitative RT-PCR. Alternatively, for example, the expression of CLDN18.2 protein on the cell surface can be determined by such methods as immunohistochemistry, FACS etc. using antibodies against CLDN18.2 protein. For example, the CLDN18.2-positive tumor cells may be Raji-CLDN18.2 tumor cells and/or CFPAC-1 tumor cells.

### DETAILED DESCRIPTION OF THE INVENTION

Chimeric Antigen Receptor

In one aspect, the present application provides a chimeric antigen receptor (CAR) that combines antibody-based specificity for a target antigen (e.g., a tumor antigen) with a T cell receptor activated intracellular domain to produce a chimeric protein that exhibits specific anti-tumor cell immune activity. The term “chimeric” as used herein describes that it consists of portions of different proteins or DNA from different sources. CAR considered by the present application comprises an extracellular domain (also referred to as a binding domain or an antigen-specific binding domain, such as a CLDN18.2 binding domain), a transmembrane domain, a costimulatory domain, and an intracellular signaling domain that binds to a specific target antigen. The main characteristics of CAR lie in their ability to specifically redirect immune effector cells to induce proliferation, cytokine production, phagocytosis, or the production of molecules capable of mediating cell death of cells expressing a target antigen in a manner independent of major histocompatibility (MHC), the ability to specifically target cells using monoclonal antibodies, soluble ligands, or cell-specific co-receptors.

In another aspect, the present application provides a CAR comprising an extracellular binding domain that specifically binds to a target antigen, including but not limited to an extracellular domain of a single-chain antibody, antibody or antigen-binding fragment thereof, tethered ligand, or co-receptor, with the target antigen being a tumor-associated antigen (TAA) or a tumor-specific antigen (TSA). TAA or TSA may be expressed on blood cancer cells or expressed on solid tumor cells. The solid tumor may be glioblastoma, non-small cell lung cancer, lung cancer other than non-small cell lung cancer, breast cancer, prostate cancer, pancreatic cancer, liver cancer, colon cancer, gastric cancer, spleen cancer, skin cancer, brain cancer other than glioblastoma, kidney cancer, thyroid cancer etc. TAA or TSA may be selected from the following group: alpha folate receptor, 5T4, αvβ 6 integrin, BCMA, B7-H3, B7-H6, CAIX, CD19, CD20, CD22, CD30, CD33, CD44, CD44v6, CD44v7/8, CD70, CD79a, CD79b, CD123, CD138, CD171, CEA, CSPG4, EGFR, EGFR family including ErbB2(HER2), EGFRvIII, EGP2, EGP40, EPCAM, EphA2, EpCAM, FAP, fetal AchR, FRα, GD2, GD3, ′phosphatidylinositol glycan-3 (GPC3), HLA-A1+MAGE1, HLA-A2+MAGE1, HLA-A3+MAGE1, HLA-A1+NY-ESO-1, HLA-A2+NY-ESO-1, HLA-A3+NYESO-1, IL-11Rα, IL-13Rα2, λ, Lewis-Y, κ, mesothelin, Muc1, Muc16, NCAM, NKG2D ligands, NY-ESO-1, PRAME, PSCA, PSMA, ROR1, SSX, survivin, TAG72, TEM, and VEGFR2.

In another aspect, the present application provides a CAR comprising a transmembrane domain that fuses an extracellular binding moiety with an intracellular signaling domain and anchors the CAR to the plasma membrane of an immune effector cell. The transmembrane domain may be derived from natural, synthetic, semi-synthetic or recombinant sources. Exemplary transmembrane domains may be derived from the following sources (i.e., include at least the following transmembrane regions): the α, β, or ζ chains of T cell receptor, CD3ε, CD3ζ, CD4, CD5, CD9, CD 16, CD22, CD27, CD28, CD33, CD37, CD45, CD64, CD80, CD86, CD134, CD137 and CD154.

In another aspect, the present application provides a CAR comprising a costimulatory signaling domain that acts in an antigen-independent manner to provide a secondary or costimulatory signal. A CAR may contain one or more “costimulatory signaling domain”. For example, the costimulatory signaling domains include the costimulatory signaling domains of CD27, CD28, 4-1BB (CD137), OX40 (CD134), CD30, CD40, PD-1, ICOS (CD278), CTLA4, LFA-1, CD2, CD7, LIGHT, TRIM, LCK3, SLAM, DAP10, LAG3, HVEM and NKD2C as well as CD83.

In another aspect, the present application provides a CAR comprising an intracellular signaling domain. The intracellular signaling domain is involved in the transduction of information effective for CAR binding to a target antigen into the interior of an immune effector cell to elicit effector cell functions such as activation, cytokine production, proliferation, and cytotoxic activity, including the release of cytotoxic factors to CAR-bound target cells or other cellular responses triggered by antigens that bind to the extracellular CAR domain, for example, the intracellular signaling domains of TCRζ, FcRγ, FcRβ, CD37, CD36, CD3E, CD3ζ, CD22, CD79a, CD79b, and CD66d.

In another aspect, the present application provides a CAR comprising one or more extracellular binding domains that specifically bind to a target antigen and one or more transmembrane domains, wherein the C-terminus of the extracellular binding domain that specifically binds to the target antigen is directly or indirectly linked to the N-terminus of the transmembrane domain, and the linkage may be achieved either directly via a peptide bond or via a linker.

In another aspect, the present application provides a CAR comprising one or more transmembrane domains and one or more costimulatory domains, wherein the C-terminus of the transmembrane domain is directly or indirectly linked to the N-terminus of the costimulatory domain, and the linkage may be achieved either directly via a peptide bond or via a linker.

In another aspect, the present application provides a CAR comprising one or more costimulatory domains and one or more intracellular signaling domains, wherein the C-terminus of the costimulatory domain is directly or indirectly linked to the N-terminus of the intracellular signaling domain, and the linkage may be achieved either directly via a peptide bond or via a linker.

In another aspect, the present application provides a CAR comprising one or more extracellular binding domains that specifically bind to a target antigen, one or more transmembrane domains, and one or more costimulatory domains, wherein the C-terminus of the extracellular binding domain that specifically binds to the target antigen is directly or indirectly linked to the N-terminus of the transmembrane domain independently, the C-terminus of the transmembrane domain is directly or indirectly linked to the N-terminus of the costimulatory domain independently, and the linkage may be directly achieved via a peptide bond or via a linker.

In another aspect, the present application provides a CAR comprising one or more transmembrane domains, one or more costimulatory domains, and one or more intracellular signaling domains, wherein the C-terminus of the transmembrane domain is directly or indirectly linked to the N-terminus of the costimulatory domain independently, the C-terminus of the costimulatory domain is directly or indirectly linked to the N-terminal of the intracellular signaling domain independently, and the linkage may be achieved either directly via a peptide bond or via a linker.

In another aspect, the present application provides a CAR comprising one or more extracellular binding domains that specifically bind to a target antigen, one or more transmembrane domains, one or more costimulatory domains, and one or more intracellular signaling domains, wherein the C-terminus of the extracellular binding domain that specifically binds to the target antigen is directly or indirectly linked to the N-terminus of the transmembrane domain independently, the C-terminus of the transmembrane domain is directly or indirectly linked to the N-terminus of the costimulatory domain independently, the C-terminus of the costimulatory domain is directly or indirectly linked to the N-terminus of the intracellular signaling domain independently, and the linkage may be achieved either via a peptide bond or via a linker.

In another aspect, the present application provides a CAR comprising an extracellular binding domain that specifically binds to a target antigen, a transmembrane domain, a costimulatory domain, and an intracellular signaling domain, wherein the C-terminus of the extracellular binding domain that specifically binds to the target antigen is directly or indirectly linked to the N-terminus of the transmembrane domain independently, the C-terminus of the transmembrane domain is directly or indirectly linked to the N-terminus of the costimulatory domain independently, the C-terminus of the costimulatory domain is directly or indirectly linked to the N-terminus of the intracellular signaling domain independently, and the linkage may be achieved via a peptide bond.

Fusion Protein or Fragment Thereof

In one aspect, the present application provides a fusion protein or fragment thereof, including a fusion polypeptide and fragment thereof. A fusion protein refers to a polypeptide comprising at least two, three, four, five, six, seven, eight, nine, or ten or even more polypeptide segments. Fusion proteins are typically proteins that link C-terminus to N-terminus, but they may also be proteins that link C-terminus to C-terminus, N-terminus to N-terminus, or N-terminus to C-terminus. The polypeptides of the fusion protein may be in any order or a designated order. Fusion polypeptides or fusion proteins may also include conservatively modified variants, polymorphic variants, alleles, mutants, subsequences, and interspecific homologs as long as the desired transcriptional activity of the fusion polypeptide is preserved. The fusion polypeptide may be produced by chemical synthetic methods or by chemical bonding between the two moieties, or the fusion polypeptide may generally be prepared using other standard techniques. The ligated DNA sequence comprising the fusion polypeptide is operably linked to a suitable transcription or translation control element. In another respect, the fusion partner comprises a sequence (expression enhancer) that facilitates expression of the protein in a higher yield than the native recombinant protein. Other fusion partners may be selected to increase the solubility of the protein or to enable the protein to target the desired intracellular compartments or to facilitate transport of the fusion protein through the cell membrane.

The fusion protein may also comprise a polypeptide cleavage signal between each polypeptide domain described herein. In addition, the polypeptide site may be located in any linker peptide sequence. Exemplary polypeptide cleavage signals include polypeptide cleavage recognition sites, such as protease cleavage sites, nuclease cleavage sites (e.g., rare restriction enzyme recognition sites, self-cleaving ribozyme recognition sites) and self-cleaving viral oligopeptides (see deFelipe and Ryan, 2004. Traffic, 5 (8); 616-26). Suitable protease cleavage sites and self-cleaving peptides are known to those skilled in the art (see, for example, Ryan et al., 1997. J. Generic. Virol. 78, 699-722; Scymczak et al. (2004) Nature Biotech 0.5, 589-594). Exemplary protease cleavage sites include, but are not limited to, the following cleavage sites: NIa protease of potato Y virus (for example, tobacco etch virus protease), HC protease of potato Y virus, P1(P35) protease of potato Y virus, byovirus NIa protease, byovirus RNA-2-encoded protease, L protease of foot-and-mouth disease virus, enterovirus 2A protease, rhinovirus 2A protease, 3C protease of RNA virus (picorna), 24K protease of cowpea mosaic virus, nepovirus 24K protease, RTSV (rice tungro spherical virus) 3C-like protease, PYVF (parsnip yellow fleck virus) 3C-like protease, heparin, thrombin, factor Xa and enterokinase. In another aspect, the self-cleaving peptides include those polypeptide sequences obtained from potato Y virus and cardiovirus 2A peptide, FMDV (foot-and-mouth disease virus), equine rhinitis virus A, thosea asigna beta tetrasoma virus, and porcine teschovirus. In another aspect, the self-cleaving polypeptide site contains a 2A or 2A-like site, sequence, or domain (Donnelly et al., 2001. J. Gen. Virol 0.82:1027-1041).

Nucleic Acid Molecule, Vector and Cell

In one aspect, the present application provides one or more nucleic acid molecules that can encode a fusion protein or fragment thereof as described herein. For example, each of the one or more nucleic acid molecules may encode the entire fusion protein or a portion thereof. The nucleic acid molecules described herein may be isolated. For example, it may be produced or synthesized by (i) amplification in vitro, such as by polymerase chain reaction (PCR) amplification, (ii) by clonal recombination, (iii) purification, such as by enzymatic cleavage and gel electrophoresis fractionation, or (iv) synthesis, such as by chemical synthesis. The isolated nucleic acid may be a nucleic acid molecule prepared by recombinant DNA technology. In the present application, nucleic acids encoding the antibodies, antigen-binding fragments thereof, may be prepared by a variety of methods known in the art including, but not limited to, restriction fragment manipulation or overlap extension PCR with synthetic oligonucleotides as described by Sambrook et al. (Molecular Cloning, A Laboratory Manual, Cold Spring Harbor Laboratory Press, Cold Spring Harbor, N.Y., 1989) and Ausube et al. (Current Protocols in Molecular Biology, Greene Publishing and Wiley-Interscience, New York N.Y, 1993).

In another aspect, the present application provides one or more vectors comprising one or more nucleic acid molecules described herein. Each vector may contain one or more of the nucleic acid molecules. In addition, other genes may be included in the vector, such as marker genes that allow selection of the vector in appropriate host cells and under appropriate conditions. Furthermore, the vector may further comprise an expression control element that allows correct expression of the coding region in a suitable host. Such control elements are well known to those skilled in the art and may include, for example, promoters, ribosome binding sites, enhancers and other control elements that regulate gene transcription or mRNA translation etc. The expression control sequence may be an adjustable element and the specific structure of the expression control sequence may vary depending on the function of the species or cell type, but generally includes 5′ non-transcriptional sequences and 5′ and 3′ non-translational sequences involved in transcription and translation initiation, respectively, such as TATA cassettes, capping sequences, CAAT sequences etc. For example, the 5′ non-transcriptional expression control sequence may comprise a promoter region, which may comprise a promoter sequence for transcriptional control of a functionally linked nucleic acid. The expression control sequence may further comprise an enhancer sequence or an upstream activator sequence. In the present application, suitable promoters may include, for example, promoters for SP6, T3 and T7 polymerases, human U6RNA promoters, CMV promoters and artificial hybrid promoters (e.g., CMV), wherein a portion of the promoter may be fused to a portion of a promoter of another cellular protein (e.g., human GAPDH, glyceraldehyde-3-phosphate dehydrogenase) gene, which may or may not contain additional introns. One or more nucleic acid molecules described herein may be operably linked to the expression control element. The vectors may include, for example, plasmids, cosmids, viruses, bacteriophages or other vectors commonly used in, for example, genetic engineering. For example, the vector is an expression vector.

In one aspect, the present application provides a cell that may comprise one or more nucleic acid molecules described herein and/or express a fusion protein described herein. Each type of cell or each cell may comprise one type or one of the nucleic acid molecules described herein or express one type or one of the fusion proteins described herein. Each type of cell or each cell may contain multiple (e.g., two or more) or multiple types (e.g., two or more types) of nucleic acid molecules described herein or express multiple (e.g., two or more) or multiple types (e.g., two or more types) of fusion proteins described herein. For example, the nucleic acid molecules described herein can be introduced into the cells, such as eukaryotic cells, cells from plants, fungal or yeast cells etc. The nucleic acid molecules described herein may be introduced into the cells by methods known in the art, such as electroporation, lipofectine transfection, lipofectamin transfection etc.

Pharmaceutical Composition

In one aspect, the present application provides a pharmaceutical composition that may comprise a fusion protein or fragment thereof, a nucleic acid molecule, a vector, a host cell, and optionally a pharmaceutically acceptable adjuvant described herein. The pharmaceutical composition of the present application may comprise a prophylactically and/or therapeutically effective dosage of the antibody, an antigen-binding fragment thereof. The prophylactically and/or therapeutically effective dosage is a dose required to be capable of preventing and/or treating (at least partially treating) a disease or disorder and/or any complications thereof in a subject having or at risk for disease development. The pharmaceutically acceptable adjuvant is non-toxic to the recipient at the dose and concentration used and may include buffers such as phosphates, citrates and other organic acids; antioxidant, including ascorbic acid and methionine; preservative (such as octadecyl dimethyl benzyl ammonium chloride, hexamethonium chloride, benzalkonium chloride, benzethonium chloride, benzethonium chloride, phenol, butanol, or benzyl alcohol; alkyl paraben, such as methyl or propyl paraben; catechol; resorcinol; cyclohexanol; 3-pentanol and m-cresol); low-molecular-weight (less than about 10 residues) polypeptides; protein, such as serum albumin, gel or immunoglobulin; hydrophilic polymer such as polyvinylpyrrolidone; amino acid such as glycine, glutamine, aspartic acid, histidine, arginine or lysine; monosaccharides, disaccharides, and other carbohydrates, including glucose, mannose, or dextrin; chelate agents, such as EDTA; saccharides such as sucrose, mannitol, trehalose or sorbitol; salt-forming counter-ions, such as sodium ion; metal complex (e. g., Zn-protein complexes); and/or nonionic surfactants such as TWEEN™, PLURONICS™, or polyethylene glycol (PEG). The pharmaceutical composition of the present application may also contain more than one active compound, typically those having complementary activities that do not exert adverse effect on each other. The type and effective dosage of such drugs depends on, for example, the amount and type of antagonist present in the formulation, as well as clinical parameters of the subject.

Treatment of Tumors

In one aspect, the present application provides methods for inhibiting tumor growth and/or killing tumors. For example, pharmaceutical composition of the present application may inhibit or delay the development or progression of a disease, may reduce tumor size (or even substantially eliminate tumors), and/or may mitigate and/or stabilize disease states. Examples of tumors include, but are not limited to, lymphoma, blastoma (including medulloblastoma and retinoblastoma), sarcoma (including liposarcoma and synovial sarcoma), neuroendocrine tumor (including carcinoid tumor, gastrinoma and islet cell carcinoma), mesothelioma, schwannoma (including acoustic neuroma), meningioma, adenocarcinoma and melanoma. The term “tumor” further includes “solid tumor”, which refers to a tumor selected from the following group: gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine cancer, salivary adenocarcinoma, renal cancer, prostate cancer, vulvar cancer, thyroid cancer, hepatic carcinoma, anal cancer, penile cancer, testicular cancer, esophageal cancer, biliary cancer, and head and neck cancer. The “tumor” may be gastric cancer, renal cancer, pancreatic cancer, and/or lymphatic cancer.

Hinge Region, Synergistic Domain, Costimulatory Synergistic Domain and Chemotactically Synergistic Domain

In one aspect, the “hinge region” or “hinge domain” as provided herein refers to two adjacent domains connecting a CAR protein. For example, extracellular domain and transmembrane domain constitutes a part of a CAR. The hinge region may be located between the various domains of the CAR, and the hinge region is added for proper spacing and conformation of the molecule. The CAR considered by the present application may comprise 1, 2, 3, 4 or 5 or more hinge regions and the length of the hinge region may range from about 1 to about 25 amino acids, from about 5 to about 20 amino acids, or from about 10 to about 20 amino acids, or any amino acid of intermediate length. The hinge region may be derived from natural, synthetic, semi-synthetic or recombinant sources and it may comprise a naturally occurring immunoglobulin hinge region or an altered amino acid sequence of an immunoglobulin hinge region.

In another aspect, one or more hinge regions of the present application may be located at any one or more locations between the extracellular binding domain, transmembrane domain, costimulatory domain, and/or intracellular signaling domain of a CAR that specifically binds to a target antigen. For example, one or more hinge regions may be located between an extracellular binding domain that specifically binds to a target antigen and a transmembrane domain, between a transmembrane domain and a costimulatory domain, and/or between a costimulatory domain and an intracellular signaling domain. For example, a hinge region may be located between an extracellular binding domain that specifically binds to a target antigen and a transmembrane domain.

In another aspect, the linkage of the hinge region to each individual domain of CAR may be achieved via the direct or indirect linkage of C-terminus of each domain of CAR to the N-terminus of the hinge region, wherein the linkage may be achieved directly via a peptide bond, or via a linker. For example, the C-terminus of the extracellular binding domain that specifically binds to the target antigen is linked to the N-terminus of the hinge region via a peptide bond.

In one aspect, the present application provides a synergistic domain, which refers to a domain capable of improving the killing capacity of a chimeric antigen receptor to tumor cells. The synergistic domain may be directly or indirectly linked to each individual domain of the CAR, for example, the C-terminus of the intracellular signaling domain is directly or indirectly linked to the N-terminus of the synergistic domain. In another aspect, the synergistic domain comprises a costimulatory synergistic domain and/or a chemotactically synergistic domain, wherein the synergistic domain comprises a protein or a functional fragment thereof selected from the following group: 4-1BB, CD28, CD27, OX40, OX40L, GITR, ICOS, CCR2, CCR5, CCR7, CCR15, CXCR2, CXCR4 and/or CXCR5.

In another aspect, the N-terminus of one or more synergistic domains of the present application may be directly or indirectly linked to the C-terminus of one or more extracellular binding domains, transmembrane domains, costimulatory domains, and/or intracellular signaling domains of a CAR that specifically binds to a target antigen. For example, one or more synergistic domains may be directly or indirectly linked to an intracellular signaling domain; for example, a synergistic domain may be directly or indirectly linked to an intracellular signaling domain. The linkage may be directly achieved via a peptide bond or via a linker. For example, the C-terminus of the intracellular signaling domain is linked to the N-terminus of the synergistic domain via a linker.

In another aspect, the present application provides a costimulatory synergistic domain, which refers to a domain capable of enhancing an effective response of lymphocytes to an antigen and/or enhancing lymphocyte proliferation capacity. The costimulatory synergistic domain may be directly or indirectly linked to each individual domain of the CAR, for example, the C-terminus of the intracellular signaling domain is directly or indirectly linked to the N-terminus of the costimulatory synergistic domain. The optionally lymphocyte costimulatory synergistic domain is selected from the following group: the costimulatory synergistic domains of 4-1BB, CD28, CD27, OX40, OX40L, GITR and/or ICOS.

In another aspect, the N-terminus of one or more costimulatory synergistic domains of the present application may be directly or indirectly linked to the C-terminus of one or more extracellular binding domains, transmembrane domains, costimulatory domains, and/or intracellular signaling domains of a CAR that specifically binds to a target antigen. For example, one or more costimulatory synergistic domain may be directly or indirectly linked to an intracellular signaling domain. For example, a costimulatory synergistic domain may be directly or indirectly linked to an intracellular signaling domain. The linkage may be directly achieved via a peptide bond or via a linker. For example, the C-terminus of the intracellular signaling domain is linked to the N-terminus of the costimulatory synergistic domain via a linker. For example, the C-terminus of the intracellular signaling domain is linked to the N-terminus of the costimulatory synergistic domain via a linker.

In another aspect, the present application provides a chemotactically synergistic domain, which refers to a domain capable of improving the killing capacity of lymphocytes to tumors and/or improving the ability of lymphocytes to regulate tumor apoptosis. The chemotactically synergistic domain may be directly or indirectly linked to each individual domain of the CAR, for example, the C-terminus of the intracellular signaling domain is directly or indirectly linked to the N-terminus of the chemotactically synergistic domain. The optionally chemotactically synergistic domain of lymphocyte is selected from the following group: chemotactically synergistic domain of CCR2, CCR5, CCR7, CCR15, CXCR2, CXCR4 and/or CXCR5.

In another aspect, the N-terminus of one or more chemotactically synergistic domains of the present application may be directly or indirectly linked to the C-terminus of one or more extracellular binding domains, transmembrane domains, costimulatory domains, and/or intracellular signaling domains of the CAR that specifically binds to a target antigen. For example, one or more chemotactically synergistic domains may be directly or indirectly linked to an intracellular signaling domain; or, for example, a chemotactically synergistic domain may be directly or indirectly linked to an intracellular signaling domain. The linkage may be directly achieved via a peptide bond or via a linker. For example, the C-terminus of the intracellular signaling domain is linked to the N-terminus of the chemotactically synergistic domain via a linker.

For example, the chimeric antigen receptor targeting CLDN18.2 of the present application comprises an extracellular binding domain, a hinge region, a transmembrane domain, a costimulatory domain, an intracellular signaling domain and a synergistic domain that specifically binds to a target antigen, wherein the C-terminus of the extracellular binding domain that specifically binds to the target antigen is directly linked to the N-terminus of the hinge region via a peptide bond, the C-terminus of the hinge region is directly linked to the N-terminus of the transmembrane domain via a peptide bond, the C-terminus of the transmembrane structure domain is directly linked to the N-terminus of the costimulatory domain via a peptide bond, the C-terminus of the costimulatory domain is directly linked to the N-terminus of the intracellular signaling domain via a peptide bond, and the C-terminus of the intracellular signaling domain is linked to the N-terminus of the synergistic domain via a linker.

For example, the chimeric antigen receptor targeting CLDN18.2 of the present application comprises an extracellular binding domain, a hinge region, a transmembrane domain, a costimulatory domain, an intracellular signaling domain and a costimulatory synergistic domain that specifically binds to a target antigen, wherein the C-terminus of the extracellular binding domain that specifically binds to the target antigen is directly linked to the N-terminus of the hinge region via a peptide bond, the C-terminus of the hinge region is directly linked to the N-terminus of the transmembrane domain via a peptide bond, the C-terminus of the transmembrane structure domain is directly linked to the N-terminus of the costimulatory domain via a peptide bond, the N-terminus of the costimulatory domain is directly linked to the N-terminus of the intracellular signaling domain via a peptide bond, and the C-terminus of the intracellular signaling domain is linked to the N-terminus of the costimulatory synergistic domain via a linker.

For example, the chimeric antigen receptor targeting CLDN18.2 of the present application comprises an extracellular binding domain, a hinge region, a transmembrane domain, a costimulatory domain, an intracellular signaling domain and a chemotactically synergistic domain that specifically binds to a target antigen, wherein the C-terminus of the extracellular binding domain that specifically binds to the target antigen is directly linked to the N-terminus of the hinge region via a peptide bond, the C-terminus of the hinge region is directly linked to the N-terminus of the transmembrane domain via a peptide bond, the C-terminus of the transmembrane domain is directly linked to the N-terminus of the costimulatory domain via a peptide bond, the C-terminus of the costimulatory domain is directly linked to the N-terminus of the intracellular signaling domain via a peptide bond, and the C-terminus of the intracellular signaling domain is linked to the N-terminus of the chemotactically synergistic domain via a linker.

Preparation, Method and Use

In one aspect, the present application provides a method for preparing a fusion protein or fragment thereof. The method may include culturing the cells of the present application under conditions such that the fusion protein or fragment thereof is expressed, for example, an appropriate medium, an appropriate temperature, a proper culture duration etc. can be used, as known by those generally skilled in the art. In some cases, the method may further comprise the step of isolating and/or purifying the fusion protein or fragment thereof. For example, affinity chromatography may be performed using protein G-agarose or protein A-agarose, and the fusion protein or fragments thereof described in the present application may also be purified and isolated by gel electrophoresis and/or high-performance liquid chromatography (HPLC) etc.

In another aspect, the present application provides a method for treating cancer in a subject, inhibiting tumor growth and/or inhibiting tumor cell proliferation in a subject, including administering to the subject or the tumor cell in need the fusion protein or fragment thereof and/or the pharmaceutical composition described herein. Administration of the aforementioned items may be performed using any suitable method and such suitable methods include, for example, in an intravenous manner, in an intramuscular manner, in a subcutaneous manner, in an intradermal manner, in a transdermal manner, in an intra-arterial manner, in an intraperitoneal manner, in an intra-lesional manner, in an intracranial manner, in an intra-articular manner, in an intra-prostatic manner, in an intrapleural manner, in an intratracheal manner, in an intrathecal manner, in an intravaginal manner, in an intrarectal manner, in an intratumoral manner, in an intraperitoneal manner, in a subconjunctival manner, in an intracapsular manner, in a mucosal manner, in a pericardial manner, in an umbilical manner, in an intraocular manner, in an orbital manner, in an oral manner, in a topical manner, in a transdermal manner, in an intravitreal manner (e.g., by intravitreal injection), by eye drops, by inhalation, by injection, by implantation, by infusion, by continuous infusion, by topical perfusion of directly bathing target cells, by catheter, by lavage, in the form of a cream, or in the form of a lipid composition. The compositions used in the methods described in the present application may also be administered systemically or topically. However, the method of administration may vary depending on the various factors (e.g., the compound or composition administered and the severity of the condition, disease, or condition being treated). For example, an anti-cancer therapy (e.g., anti-CLDN18.2 antibody) is administered intravenously, intramuscularly, in a subcutaneous, topical, oral, percutaneous, intraperitoneal, intraorbital, intrathecal, intracardiac, or intranasal manner, or by implantation, by inhalation. Depending in part on whether the administration is given on a short-term or long-term basis, the administration may be performed by any suitable route, such as by injection, such as intravenous or subcutaneous injection. The present application covers a variety of dosing schedules including, but not limited to, a single administration or multiple administrations, bolus administrations, and pulse-based infusions within various time points.

In another aspect, that present application provides the use of the fusion protein or fragment thereof and/or the pharmaceutical composition in the preparation of a medicament which is used for treating a cancer, inhibiting tumor growth and/or inhibiting tumor cell proliferation. The tumor or cancer may include lymphoma and/or pancreatic cancer, or it may be a tumor or cancer with abnormal expression of CLDN18.2.

The fusion protein or fragments thereof and/or the pharmaceutical composition described herein may be formulated, administered and used in a manner consistent with good medical practice. Considerations in this case include the particular disorder being treated, the particular mammal being treated, the clinical pathology of the individual patient, the etiology of the disorder, the agent delivery site, the method of administration, the administration schedule, and other factors known to the medical practitioner. Therapeutic agents (e.g., anti-CLDN18.2 antibodies) are not required, but are optionally formulated and/or administered together with one or more agents currently used to prevent or treat conditions of interest. The effective dosage of such other agents depends on the amount of therapeutic agent (e.g., anti-CLDN18.2 antibody) present in the formulation, the type of disorder or treatment, and other factors discussed above. These agents may generally be administered at any dose empirically/clinically determined to be appropriate and by any route empirically/clinically determined to be appropriate. The dosage of antibody administered in a combination treatment may be reduced compared to a monotherapy treatment. The disease progression under such therapy can be easily monitored using conventional techniques.

Enhancing Tumor Cell Killing Capacity

In one aspect, the present application provides a method for enhancing the killing capacity of a chimeric antigen receptor and/or lymphocyte expressing the chimeric antigen receptor to tumor cells, comprising the steps of linking the chimeric antigen receptors to a synergistic domain, wherein the synergistic domain comprises a protein or a functional fragment thereof selected from the following group: 4-1BB, CD28, CD27, OX40, OX40L, GITR, ICOS, CCR2, CCR5, CCR7, CCR15, CXCR2, CXCR4, and/or CXCR5.

In another aspect, the present application provides a method for enhancing the proliferation capability of a chimeric antigen receptor and/or lymphocyte expressing the chimeric antigen receptor, comprising the steps of linking the chimeric antigen receptor targeting CLDN18.2 to a synergistic domain, wherein the synergistic domain comprises a protein or a functional fragment thereof selected from the following group: 4-1BB, CD28, CD27, OX40, OX40L, GITR, and/or ICOS.

In another aspect, the present application provides a method for enhancing the regulation of tumor apoptosis by chimeric antigen receptors and/or lymphocyte expressing the chimeric antigen receptor, comprising the steps of linking the chimeric antigen receptor to a synergistic domain, wherein the synergistic domain comprises a protein selected from the following group: CCR2, CCR5, CCR7, CCR15, CXCR2, CXCR4, and/or CXCR5.

Without intending to be bound by any theory available, the following embodiments are merely illustrative of the fusion protein, method of preparation, use, etc. of the present application and are not intended to limit the scope of the invention of the present application.

### Examples

In the present application, the correspondence of the names of the binding target, single-chain antibody (scFv, single chain fragment variable), chimeric antigen receptor (CAR), vector, and cell are shown in Table 1.

TABLE 1
Correspondence of the names of the binding target, single-chain
antibody (scFv, single chain fragment variable), chimeric antigen
receptor (CAR), vector and cell of the present application

| Binding | scFv Nucleotide | CAR Amino Acid | | |
|---|---|---|---|---|
| Target | Sequence | Sequence | Vector | Cell |
| CLDN18.2 | scFvAb10 | Ab10BBZ | Ab10BBZ virus | Ab10BBZ CAR-T |
| | (SEQ ID NO: 1) | (SEQ ID NO: 14) | | cell |
| CD20 | scFv 20 | 20BBZ | 20BBZ virus | 20BBZ CAR-T cell |
| | (SEQ ID NO: 8) | (SEQ ID NO: 15) | | |
| CLDN18.2 | scFv Ab362 | Ab362BBZ | Ab362BBZ virus | Ab362BBZ CAR-T |
| | (SEQ ID NO: 2) | (SEQ ID NO: 16) | | cell |
| CLDN18.2 | scFvAb10 | Ab10BBZ-OX40 | Ab10BBZ-OX40 | Ab10BBZ-OX40 |
| | (SEQ ID NO: 1) | (SEQ ID NO: 17) | virus | CAR-T cell |
| CLDN18.2 | scFvAb10 | Ab10BBZ-OX40L | Ab10BBZ-OX40L | Ab 10BBZ-OX40L |
| | (SEQ ID NO: 1) | (SEQ ID NO: 18) | virus | CAR-T cell |
| CLDN18.2 | scFvAb362 | Ab362BBZ-OX40 | Ab362BBZ-OX40 | Ab362BBZ-OX40 |
| | (SEQ ID NO: 2) | (SEQ ID NO: 19) | virus | CAR-T cell |
| CLDN18.2 | scFvAb362 | Ab362BBZ-OX40L | Ab362BBZ-OX40L | Ab362BBZ-OX40L |
| | (SEQ ID NO: 2) | (SEQ ID NO: 20) | virus | CAR-T cell |
| CLDN18.2 | scFvAb10 | Ab10BBZ-CXCR5 | Ab10BBZ- CXCR5 | Ab10BBZ- CXCR5 |
| | (SEQ ID NO: 1) | (SEQ ID NO: 21) | virus | CAR-T cell |
| CLDN18.2 | scFvAb10 | Ab10BBZ-CCR7 | Ab10BBZ-CCR7 | Ab10BBZ-CCR7 |
| | (SEQ ID NO: 1) | (SEQ ID NO: 22) | virus | CAR-T cell |

### Example 1 Preparation of Anti-CLDN18.2 CAR-T Cells

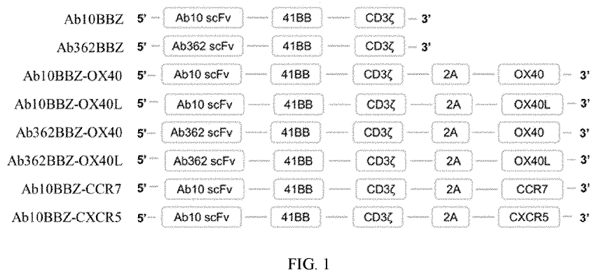

Non-synergistic CAR (Ab10BBZ and Ab362BBZ with the structures shown in FIG. 1) targeting CLDN18.2 and the control CAR (20BBZ) were prepared. The following sequences were artificially synthesized: scFv Ab10 (amino acid sequence SEQ ID NO: 28, nucleotide sequence SEQ ID NO: 1), scFv Ab362 (amino acid sequence SEQ ID NO: 29, nucleotide sequence SEQ ID NO: 2), hinge region (amino acid sequence SEQ ID NO: 30, nucleotide sequence SEQ ID NO: 3), a transmembrane region (amino acid sequence SEQ ID NO: 31, nucleotide sequence SEQ ID NO: 4), a 4-1BB costimulatory factor (amino acid sequence SEQ ID NO: 32, nucleotide sequence SEQ ID NO: 5), and a CD3ζ intracellular signaling domain (amino acid sequence SEQ ID NO: 33, nucleotide sequence SEQ ID NO: 6), wherein a hinge region, a transmembrane region, a 4-1BB costimulatory factor and a CD3ζ intracellular signaling domain were connected in an end-to-end manner to obtain BBZ, and the nucleotide sequence was shown in SEQ ID NO: 7. Meanwhile, scFv 20 was constructed as a control, and its nucleotide sequence was shown in SEQ ID NO: 8. The scFv Ab10 (amino acid sequence SEQ ID NO: 28, nucleotide sequence SEQ ID NO: 1) and BBZ (nucleotide sequence SEQ ID NO: 7) that can specifically bind to CLDN18.2 were added with XbaI and BamHI restriction sites at both ends to clone the pCDH-MSCVEF vector using overlap PCR. PCR amplification was carried out, and XbaI restriction site (including protective bases), scFvAb10, hinge region, transmembrane region, 4-1BB costimulatory factor, CD3 ζ intracellular signaling domain, and BamHI restriction site were sequentially carried on the 5′ end by extension PCR, and the CAR: Ab10BBZ (amino acid sequence SEQ ID NO: 14) was obtained by PCR amplification.

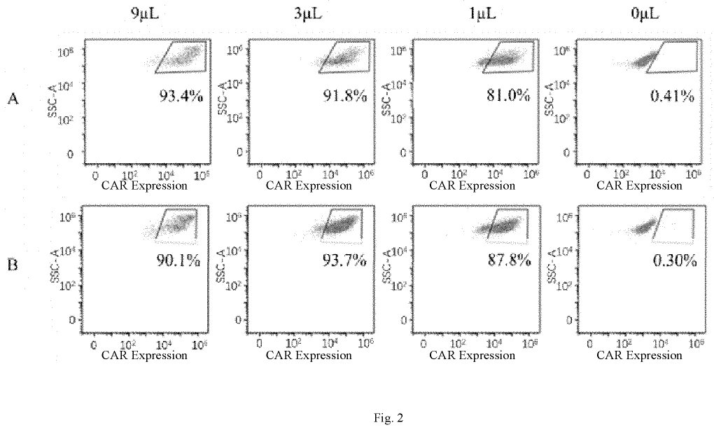

Non-synergistic anti-CLDN18.2 CAR-T virus (Ab10BBZ virus and Ab362BBZ virus) and control CAR-T virus (20BBZ virus) were prepared. The correctly sequenced clones were extracted with endotoxin-free NucleoBond Xtra Midi Plus EF kit, and then transfected into 293 cells together with lentivirus packaging plasmid (VSV-g, pMD Gag/pol or RSV-REV). The cells were then incubated at 37° C. for 48 hours in 5% CO₂ and the supernatant was collected. After filtration through 0.45 μm membrane, the supernatant were centrifuged at 25000 RPM for 2 hours using a Beckman ultracentrifuge and a SW28 centrifuge head to concentrate and obtain the pCDH-MSCVEF-Ab10BBZ virus (abbreviated as Ab10BBZ virus) for subsequent manufacturing of CAR-T cells. Meanwhile, the control pCDH-MSCVEF-20BBZ virus and pCDH-MSCVEF-Ab362BBZ virus (abbreviated as 20BBZ virus and Ab362BBZ virus) were produced using the same procedure as for the preparation of Ab10BBZ virus, and the resulting virus was infected with 293 cells, then the viral titer was measured by flow cytometry using anti-mouse Fab antibody (Jackson ImmunoResearch #115-605-006). Shown in FIGS. 2A-2B are the results obtained with flow cytometry when 1 μL, 3 μL, and 9 μL of the virus were added, with no virus-added cell as a blank control. The results showed that the expression level of CAR: 20BBZ (amino acid sequence SEQ ID NO: 15) and Ab362BBZ (amino acid sequence SEQ ID NO: 16) in CAR increased with an increase in the dose of virus added.

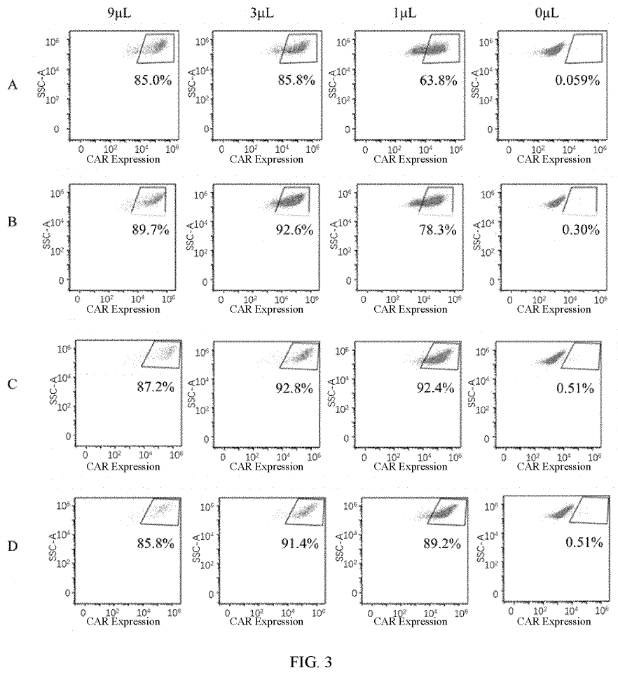

Similarly, a costimulatory synergistic CAR targeting CLDN18.2 (Ab10BBZ-OX40, Ab10BBZ-OX40L, Ab362BBZ-OX40 and Ab362BBZ-OX40L with the structures shown in FIG. 1) and a costimulatory synergistic anti-CLDN18.2 CAR-T virus (Ab10BBZ-OX40 virus, Ab10BBZ-OX40L virus, Ab362BBZ-OX40 virus and Ab362BBZ-OX40L virus) were prepared. The stop codon was removed from Ab10BBZ, Ab362BBZ, fragment 2A (amino acid sequence SEQ ID NO: 27, nucleotide sequence SEQ ID NO: 9), OX40 (amino acid sequence SEQ ID NO: 23, nucleotide sequence SEQ ID NO: 10) or OX40L (amino acid sequence SEQ ID NO: 24, nucleotide sequence SEQ ID NO: 11) was ligated. Overlap PCR, molecular cloning and virus production was performed to obtain pCDH-MSCVEF-Ab10BBZ-OX40 virus, pCDH-MSCVEF-Ab10BBZ-OX40L virus, pCDH-MSCVEF-Ab362BBZ-OX40 virus and pCDH-MSCVEF-Ab362BBZ-OX40L virus (abbreviated as Ab10BBZ-OX40 virus, Ab10BBZ-OX40L virus, AB362BBZ-OX40 virus and AB362BBZ-OX40L virus). Similarly, viral titer was measured by flow cytometry. Shown in Figures. 3A-3D are the results obtained with flow cytometry when 1 μL, 3 μL, and 9 μL of the virus were added, with no virus-added cell as a blank control. The results showed that the expression level of CAR: Ab10BBZ-OX40 (amino acid sequence SEQ ID NO: 17), Ab10BBZ-OX40L (amino acid sequence SEQ ID NO: 18), Ab362BBZ-OX40 (amino acid sequence SEQ ID NO: 19) and Ab362BBZ-OX40L (amino acid sequence SEQ ID NO: 20) in CAR increased with an increase in the dose of virus added.

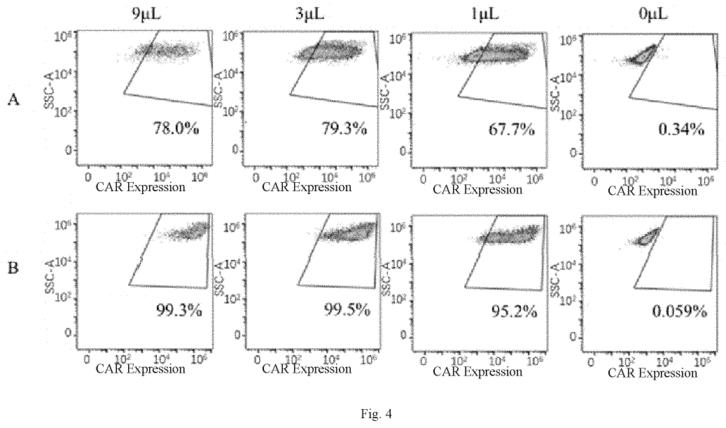

Similarly, chemotactically synergistic anti-CLDN18.2 CAR (Ab10BBZ-CCR7 and Ab10BBZ-CXCR5 with the structures shown in FIG. 1) and chemotactically synergistic CAR-T virus (Ab10BBZ-CCR7 virus and Ab10BBZ-CXCR5 virus) were prepared. The stop codon was removed from Ab10BBZ, Ab362BBZ, fragment 2A (amino acid sequence SEQ ID NO: 27, nucleotide sequence SEQ ID NO: 9), CCR7 (amino acid sequence SEQ ID NO: 25, nucleotide sequence SEQ ID NO: 12) or CXCR5 (amino acid sequence SEQ ID NO: 26, nucleotide sequence SEQ ID NO: 13) was ligated. Overlap PCR molecular cloning and virus production was performed by to obtain pCDH-MSCVEF-Ab10BBZ-CCR7 virus and pCDH-MSCVEF-Ab10BBZ-CXCR5 virus (abbreviated as Ab10BBZ-CCR7 virus and Ab10BBZ-CXCR5 virus). Similarly, viral titer was measured by flow cytometry. Shown in Figures. 4A-4B are the results obtained with flow cytometry when 1 μL, 3 μL, and 9 μL of the virus were added, with no virus-added cell as a blank control. The results showed that the expression level of CAR: Ab10BBZ-CCR7 (amino acid sequence SEQ ID NO: 22), Ab10BBZ-OX40L (amino acid sequence SEQ ID NO: 18), Ab362BBZ-OX40 (amino acid sequence SEQ ID NO: 19) and Ab10BBZ-CXCR5 (amino acid sequence SEQ ID NO: 21) in CAR increased with an increase in the dose of virus added.

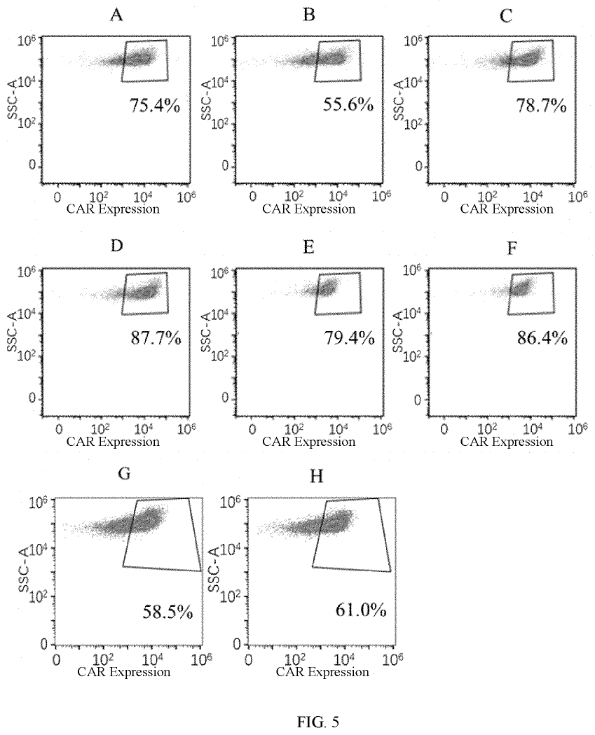

Non-synergistic anti-CLDN18.2 CAR-T cell (Ab10BBZ CAR-T cell and Ab362BBZ CAR-T cell) and control CAR-T cell (20BBZ CAR-T cell) were prepared. Human PBMC were purified using a Stemcell T-cell isolation kit (purchased from stem cell, Catalog No.: 19671) and inoculated into 96-well plates coated with anti-hCD3 (purchased from Bioxcell, Catalog No.: BE0001-2) and anti-hCD28 (purchased from Bioxcell #BE0248) and then used to infect the Ab10BBZ virus, the 20BBZ virus and the Ab362BBZ virus prepared in this example according to the MOI (multiplicity of infection, i.e., the ratio of the amount of virus to the number of cells)=10-20 after 2 days, and the cell culture was continued after 1 day in a medium changed to RPMI complete medium containing 10% FBS, IL2(50 IU/ml), IL21(4 ng/ml); after stimulation with artificially prepared antigen-presenting cells (Raji-CLDN18.2 cells after X-ray irradiation at 100Gray) or anti-hCD3 (0.1 g/ml) or anti-hCD28 (0.25 g/ml) every 6 days for 2 rounds, the cells obtained were Ab10BBZ CAR-T cells, 20BBZ CAR-T cells and Ab362BBZ CAR-T cells, then they were stained with Alexa Fluor® 647 AffiniPure F(ab′)₂ Fragment Goat Anti-Mouse IgG, Fab fragment specific antibody and determined using flow cytometry. The results as shown in FIGS. 5A and 5C indicated that the cells obtained were all CAR positive.

Similarly, costimulatory synergistic anti-CLDN18.2 CAR-T cells (Ab10BBZ-OX40 CAR-T cells, Ab10BBZ-OX40L CAR-T cells, Ab362BBZ-OX40 CAR-T cells and Ab362BBZ-OX40L Anti-CLDN18.2 CAR-T cells) were prepared. T cells derived from human PBMCs were purified, activated, and then infected and amplified by Ab10BBZ-OX40 virus, Ab10BBZ-OX40L virus, Ab362BBZ-OX40 virus and AB362BBZ-OX40L virus respectively to obtain Ab10BBZ-OX40 CAR-T cells, Ab10BBZ-OX40L CAR-T cells, Ab362BBZ-OX40 CAR-T cells and Ab362BBZ-OX40L CAR-T cells, then these cells were stained with Alexa Fluor® 647 AffiniPure F(ab′)₂ Fragment Goat Anti-Mouse IgG, Fab fragment specific antibody using flow cytometry. The results obtained are as shown in FIGS. 5B, 5D and 5E-5F, indicating that the cells obtained were all CAR positive.

Similarly, chemotactically synergistic anti-CLDN18.2 CAR-T cells (Ab10BBZ-CCR7 CAR-T cells and Ab10BBZ-CXCR5 CAR-T cells) were prepared. T cells derived from human PBMC were purified, activated, and then infected and amplified by Ab10BBZ-CCR7 virus and Ab10BBZ-CXCR5 CAR-T virus respectively to obtain Ab10BBZ-CCR7 CAR-T cells and Ab10BBZ-CXCR5 CAR-T cells, then these cells were stained with Alexa Fluor® 647 AffiniPure F(ab′)₂ Fragment Goat Anti-Mouse IgG, Fab fragment specific antibody using flow cytometry. The results obtained are as shown in FIGS. 5G-5H, indicating that the cells obtained were all CAR positive.

### Example 2 Amplification Capability of Anti-CLDN18.2 CAR-T Cells

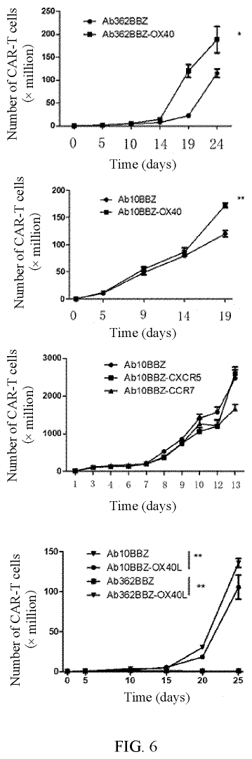

The Ab10BBZ CAR-T cells, Ab10BBZ-OX40 CAR-T cells and Ab10BBZ-OX40L CAR-T cells, Ab362BBZ CAR-T cells, Ab362BBZ-OX40 CAR-T cells and Ab362BBZ-OX40L CAR-T cells prepared in Example 1 were continuously cultured and stimulated with artificially prepared antigen-presenting cells every 6 days followed by cell counting, and the results are shown in FIG. 6. As shown in FIG. 6, Ab10BBZ-OX40 CAR-T cells and Ab10BBZ-OX40L CAR-T cells provided higher amplification capacity than Ab10BBZ CAR-T cells, and Ab362BBZ-OX40 CAR-T cells and Ab362BBZ-OX40L CAR-T cells provided higher amplification capacity than Ab362BBZ CAR-T cells.

The Ab10BBZ CAR-T cells, Ab10BBZ-CCR7 CAR-T cells and Ab10BBZ-CXCR5 CAR-T cells prepared in Example 1 were continuously cultured for 14 days and stimulated with artificially prepared antigen-presenting cells every 6 days followed by cell counting, and the results are shown in FIG. 6. As shown in FIG. 6, Ab10BBZ-CCR7 CAR-T cells and Ab10BBZ-CXCR5 CAR-T cells delivered similar in vitro amplification capacity compared with Ab10BBZ CAR-T cells.

### Example 3 Tumor Killing Capability of Anti-CLDN18.2 CAR-T Cells In Vitro

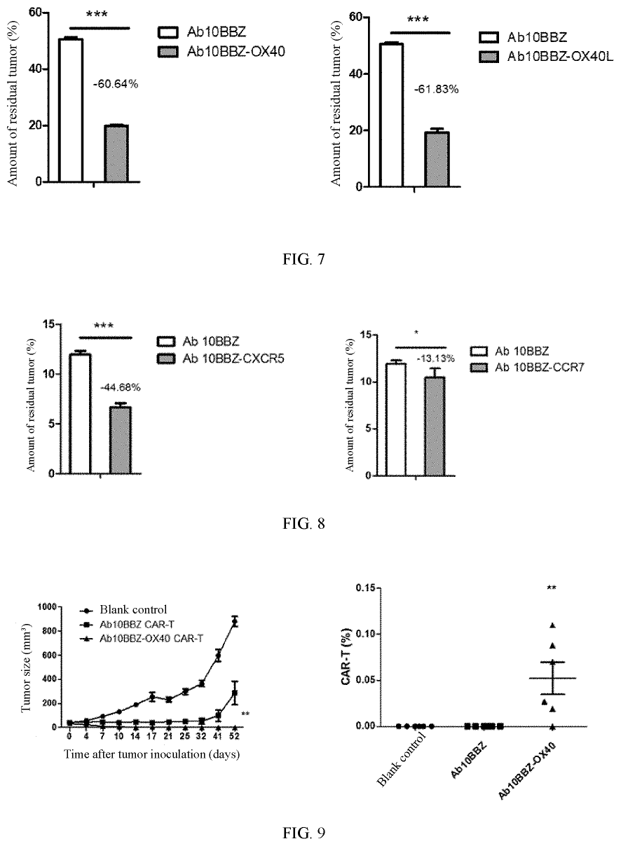

The Ab10BBZ CAR-T cells, Ab10BBZ-OX40 CAR-T cells and Ab10 BBZ-OX40L CAR-T cells prepared in Example 1 were inoculated into a 96-well plate, and CLDN18.2-positive tumor cells (Raji-CLDN18.2 tumor cells) were added using a ratio of CAR-T: tumor cells at 1:1. After 24 hours, the viability of Raji-CLDN18.2 was detected by flow cytometry. A shown in FIG. 7, the detection results obtained showed that Ab10BBZ-OX40 CAR-T cells have a more potent in vitro tumor killing capability than Ab10BBZ CAR-T cells.

The Ab10BBZ CAR-T cells, Ab10BBZ-CCR7 CAR-T cells and Ab10BBZ-CXCR5 CAR-T cells prepared in Example 1 were inoculated into a 96-well plate, and CLDN18.2-positive tumor cells (Raji-CLDN18.2 tumor cells) were added using a ratio of CAR-T: tumor cells at 1:1. After 24 hours, the viability of Raji-CLDN18.2 was detected by flow cytometry. As shown in FIG. 8, compared with Ab10BBZ CAR-T cells, Ab10BBZ-CCR7 CAR-T cells and Ab10BBZ-CXCR5 CAR-T cells could selectively kill more CLDN18.2-positive tumor cells, with the in vitro killing capacity increased by 13.1% and 44.7%, respectively.

### Example 4 Antitumor Activity of Anti-CLDN18.2 CAR-T Cells In Vivo

3×10⁶ CFPAC-1 tumor cells were subcutaneously inoculated into B-NDG mice. After 6 days, 10⁷ Ab10BBZ CAR-T cells or Ab10BBZ-OX40 CAR-T cells were administered, and PBS was administered as a blank control; then the tumor burden in the mice was measured for each group. As shown in FIG. 9 for the results obtained for each group, Ab10BBZ-OX40 CAR-T cells delivered a superior control ability in tumor burden compared with Ab10BBZ CAR-T cells. The results showed that Ab10BBZ-OX40 CAR-T cells reduced the mouse tumor size by 82.9% (3.798 mm³ to 0.646 mm³) compared to the control Ab10BBZ CAR-T cells.

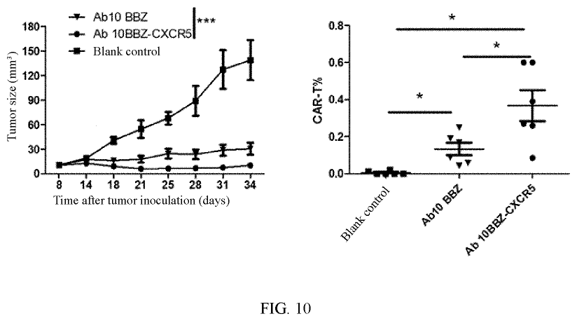

3×10⁶ CFPAC-1 tumor cells were subcutaneously inoculated into B-NDG mice. After 6 days, 10⁷ Ab10BBZ CAR-T cells or Ab10BBZ-CXCR5 CAR-T cells were administered, and PBS was administered as a blank control; then the tumor burden in the mice and the continuous proliferation of CAR-T in vivo were measured for each group. The results obtained for each group are shown in FIG. 10, respectively. As shown in FIG. 10, Ab10BBZ-CXCR5 CAR-T cells had a superior control ability in tumor burden compared with Ab10BBZ CAR-T cells. The results showed that Ab10BBZ-CXCR5 CAR-T cells reduced the mouse tumor size by 67.7% (30.98 mm³ to 10.24 mm³) compared to the control Ab10BBZ CAR-T cells, Ab10BBZ-CXCR5 CAR-T cells showed improved in vivo persistent proliferation by 2.76 folds (0.133% to 0.367%) compared to control Ab10BBZ CAR-T cells.

1. A fusion protein comprising:
a) a chimeric antigen receptor (CAR) targeting claudin 18.2 (CLDN18.2), and
b) a synergistic domain that can improve a tumor cells killing capacity of said chimeric antigen receptor targeting CLDN18.2.

2. The fusion protein according to claim 1, wherein said synergistic domain comprises a costimulatory synergistic domain, said costimulatory synergistic domain comprises a protein or a functional fragment thereof selected from the following group: OX40 and OX40L.

3. The fusion protein according to claim 2, wherein said costimulatory synergistic domain comprises an amino acid sequence as shown in any one of SEQ ID NOs: 23-24.

4. The fusion protein according to claim 1, wherein said synergistic domain comprises a chemotactically synergistic domain, said chemotactically synergistic domain comprises a protein or a functional fragment thereof selected from the following group: CCR7 and CXCR5.

5. The fusion protein according to claim 4, wherein said chemotactically synergistic domain comprises an amino acid sequence as shown in any one of SEQ ID NOs: 25-26.

6. The fusion protein according to claim 1, wherein a C-terminus of said chimeric antigen receptor targeting CLDN18.2 is directly or indirectly linked to an N-terminus of said synergistic domain.

7. The fusion protein according to claim 6, wherein said linkage is achieved via a linker.

8. (canceled)

9. The fusion protein according to claim 1, consisting of a single-chain structure.

10. The fusion protein according to claim 7, comprising said chimeric antigen receptor targeting CLDN18.2, said linker and said synergistic domain in sequence from the N-terminus to the C-terminus.

11. The fusion protein according to claim 1, wherein said chimeric antigen receptor targeting CLDN18.2 comprises a CLDN18.2-binding domain, a transmembrane domain, a costimulatory domain and an intracellular signaling domain, wherein said CLDN18.2-binding domain comprises an antibody or a fragment thereof that specifically binds to CLDN18.2.

12. The fusion protein according to claim 11, wherein said antibody is a single-chain antibody.

13. (canceled)

14. The fusion protein according to claim 11, wherein said transmembrane domain comprises a transmembrane domain derived from a protein selected from the following group: alpha, beta or zeta chain of a T cell receptor, CD28, CD3e, CD45, CD4, CD5, CD8a, CD9, CD16, CD22, CD33, CD37, CD64, CD80, CD86, CD134, CD137, and CD154.

15. (canceled)

16. The fusion protein according to claim 11, wherein said costimulatory domain comprises a costimulatory domain derived from a protein selected from the following group: CD28, 4-1BB, OX40, and ICOS.

17. (canceled)

18. The fusion protein according to claim 11, wherein said intracellular signaling domain comprises a signaling domain derived from CD3ζ.

19. (canceled)

20. The fusion protein according to claim 1, wherein said chimeric antigen receptor targeting CLDN18.2 comprises an amino acid sequence as shown in any one of SEQ ID NOs: 23-33.

21. One or more isolated nucleic acid molecules, encoding the fusion protein or a fragment thereof according to claim 1.

22. A vector, comprising the nucleic acid molecules according to claim 21.

23. A cell, expressing the fusion protein according to claim 1.

24. (canceled)

25. A pharmaceutical composition, comprising the cell according to claim 23 and a pharmaceutically acceptable adjuvant.

26. A method for treating a tumor, comprising administering the cell according to claim 23 to a subject in need thereof.

27. The method according to claim 26, wherein said tumor comprises lymphoma, gastric cancer and/or pancreatic cancer.

28. A method for improving a tumor cell killing capacity of a chimeric antigen receptor targeting CLDN18.2, comprising: linking said chimeric antigen receptor targeting CLDN18.2 to a synergistic domain, wherein said synergistic domain comprises a protein or a functional fragment thereof selected from the following group: OX40, OX40L, CCR7, and CXCR5.

29-33. (canceled)

34. A method for improving a proliferation capability of T cells comprising a chimeric antigen receptor targeting CLDN18.2, comprising: linking said chimeric antigen receptor targeting CLDN18.2 to a synergistic domain, wherein said synergistic domain comprises a protein or a functional fragment thereof selected from the following group: OX40, OX40L, CCR7, and CXCR5.

35-39. (canceled)

40. The method according to claim 34, wherein said T cells are derived from peripheral blood mononuclear cells (PBMC).


SEQUENCE LISTING (33 sequences)

SEQ ID NO: 1 - DNA, 813 bp, Artificial Sequence
  Ab10
atggagaccgacaccctcttattatgggtgctgttattatgggtccccggaagcaccggaaccggtgacatcgtcatgacccagagccccgactctctggctgtgtctctgggcgagagagccacaatcagctgcaagagcagccagtctctgctcaactccggaaaccagaagaactacctcacatggtatcagcagaagcccggccaacctcccaagctgctgatctattgggcttccacaagggaatccggcgtgcccgataggttcagcggaagcggcagcggcacagatttcacactgaccatctcctctctgcaagccgaggacgtggccgtgtactactgccagaacgactacttctaccccttcaccttcggccaaggcaccaagctggagattaagaggaccgtgggcggaggaggaagcggaggaggaggcagcggaggcggaggcagccaagtccagctcgtgcagagcggagccgaagtgaaaaaacccggctcctccgtgaaagtgagctgcaaggcctccggctatgccttctccaactatctgatcgagtgggtgaaacaagcccccggccaaggactggaatggatcggactgattaatcccggctccggcggcaccaactacaacgagaaattcaagggcaaggccaccatcaccgccgacaagagcacctccacagcctacatggagctcagctctctgagaagcgaggacacagccgtgtattactgcgctagagtgtactacggcaactccttcgcctattggggccaaggcacactggtcacagtgagcagc

SEQ ID NO: 2 - DNA, 804 bp, Artificial Sequence
  Ab362
atggagaccgacaccctcttattatgggtgctgttattatgggtccccggaagcaccggaaccggtgacatcgtgatgacccagtcccctagctctctgaccgtgacagccggagagaaagtgaccatgagctgcaagagcagccaatctctgctgaacagcggcaaccagaagaactatctgacatggtatcagcagaagcccggccaaccccccaagctgctgatttactgggccagcacaagagagagcggagtgcccgataggttcaccggcagcggcagcggcaccgatttcacactgaccatctccagcgtgcaagccgaagacctcgccgtctactactgccagaacgactacagctaccctttcaccttcggctccggcaccaaactggagatcaagggaggaggaggcagcggaggcggcggctccggcggaggcggcagccaagtgcagctgcagcagcccggcgccgagctggtgagacccggcgcctccgtgaagctgagctgcaaagctagcggctacaccttcacctcctactggatcaactgggtgaagcagagacccggccaaggactggagtggatcggcaacatctacccctccgactcctacaccaactacaaccagaagtttaaggacaaggccacactgaccgtggacaagtcctccagcaccgcctacatgcagctgtcctcccctacctccgaggacagcgccgtgtactactgcacaagaagctggaggggcaacagctttgactactggggccaaggcaccacactgaccgtgagcagc

SEQ ID NO: 3 - DNA, 135 bp, Artificial Sequence
  hinge region
acaaccacccccgctcccagacctcctacacccgctcccaccattgccagccagcctctctctttaagacccgaggcttgtaggcccgctgctggaggagccgtgcacacaaggggactggactttgcttgtgat

SEQ ID NO: 4 - DNA, 72 bp, Artificial Sequence
  transmembrane region
atctatatctgggcccctctggctggaacttgtggagtcctcttattatctttagtgatcactttatactgt

SEQ ID NO: 5 - DNA, 126 bp, Artificial Sequence
  co-stimulatory region
aagaggggtcgtaagaagttattatacatcttcaagcagcccttcatgaggcccgtccaaaccacccaagaagaggacggatgtagctgtaggtttcccgaggaggaggagggaggctgcgaatta

SEQ ID NO: 6 - DNA, 336 bp, Artificial Sequence
  intracellular signaling transduction domain
cgtgtcaagttctccagaagcgccgatgcccccgcttaccaacaaggtcagaaccagctgtacaatgagctgaatctgggcagaagagaagagtacgacgtgctggataagaggaggggtcgtgaccccgaaatgggaggcaagcccagaagaaaaaacccccaagaaggactctacaacgagctgcaaaaggataagatggctgaggcctattccgagattggcatgaagggcgagagaaggagaggcaagggccacgacggtttatatcaaggtctctccaccgccaccaaggacacatacgatgctctgcacatgcaagctctgccccccaga

SEQ ID NO: 7 - DNA, 678 bp, Artificial Sequence
  BBZ
gcggccgctacaaccacccccgctcccagacctcctacacccgctcccaccattgccagccagcctctctctttaagacccgaggcttgtaggcccgctgctggaggagccgtgcacacaaggggactggactttgcttgtgatatctatatctgggcccctctggctggaacttgtggagtcctcttattatctttagtgatcactttatactgtaagaggggtcgtaagaagttattatacatcttcaagcagcccttcatgaggcccgtccaaaccacccaagaagaggacggatgtagctgtaggtttcccgaggaggaggagggaggctgcgaattacgtgtcaagttctccagaagcgccgatgcccccgcttaccaacaaggtcagaaccagctgtacaatgagctgaatctgggcagaagagaagagtacgacgtgctggataagaggaggggtcgtgaccccgaaatgggaggcaagcccagaagaaaaaacccccaagaaggactctacaacgagctgcaaaaggataagatggctgaggcctattccgagattggcatgaagggcgagagaaggagaggcaagggccacgacggtttatatcaaggtctctccaccgccaccaaggacacatacgatgctctgcacatgcaagctctgccccccaga

SEQ ID NO: 8 - DNA, 807 bp, Artificial Sequence
  20
gaattcgccgccaccatggagaccgacaccctcttattatgggtgctgttattatgggtccccggaagcaccggaaccggtcagatcgtgctgagccagagccccgccattctgtccgccagccccggcgagaaggtgaccatgacatgcagagcctcctcctccgtgagctacatccactggttccagcagaagcccggctcctcccctaagccttggatctacgccaccagcaatctggctagcggagtgcccgtgagattcagcggcagcggaagcggaaccagctactctctgaccatcagcagagtggaggccgaggacgccgctacctactactgtcagcagtggaccagcaaccctcctaccttcggcggcggcaccaagctggagatcaagggcggaggaggctccggaggtggaggttctggcggtggaggttcccaagtgcagctgcagcagcccggcgctgagctggtgaaacccggcgcttccgtgaagatgagctgcaaggccagcggctacaccttcaccagctacaacatgcactgggtgaagcagacccccggcagaggactggaatggatcggcgccatttaccccggcaacggcgatacctcctacaaccagaagttcaagggcaaggctacactgaccgccgacaagagcagcagcaccgcctacatgcagctgagctctctgaccagcgaggacagcgccgtgtactactgcgctagaagcacctactacggcggcgactggtacttcaacgtgtggggagccggcacaaccgtgacagtgtccgcc

SEQ ID NO: 9 - DNA, 66 bp, Artificial Sequence
  2A
ggctccggcgccacaaacttctctttactgaagcaagctggagacgtggaggagaaccccggtcct

SEQ ID NO: 10 - DNA, 834 bp, Artificial Sequence
  OX40
atgtgtgtgggagccagaagactgggaagaggaccttgcgctgctctgctgctgctgggactgggactgtccacagtgaccggactgcactgcgtgggcgacacataccccagcaacgatagatgctgccacgagtgtagacccggcaatggcatggtgtccagatgctctagaagccagaatacagtgtgcagaccttgcggccccggcttttacaacgatgtggtgtccagcaagccttgcaagccttgcacatggtgtaatctgaggtccggcagcgagaggaaacaactgtgcaccgccacccaagacacagtctgcagatgtagagctggcacccagcctctggacagctataaacccggcgtggactgtgctccttgccctcccggccatttcagccccggagacaaccaagcttgcaagccttggacaaactgcacactggccggaaagcacacactgcaacccgccagcaacagctccgacgccatttgcgaagatagagaccctcccgctacacagcctcaagagacacaaggcccccccgctagacctatcaccgtccagcccacagaggcttggcctagaaccagccaaggccctagcacaagacccgtggaggtccccggcggaagagctgtggccgccattctgggcctcggactggtgctcggactgctcggacctctggctattctgctggctctgtatctgctgaggagagaccagaggctgcctcccgacgcccataaacctcccggcggcggcagctttagaacccctattcaagaggagcaagccgacgcccatagcacactggccaagatctga

SEQ ID NO: 11 - DNA, 552 bp, Artificial Sequence
  OX40L
atggaaagggtccaacccctggaagagaatgtgggaaatgcagccaggccaagattcgagaggaacaagctattgctggtggcctctgtaattcagggactggggctgctcctgtgcttcacctacatctgcctgcacttctctgctcttcaggtatcacatcggtatcctcgaattcaaagtatcaaagtacaatttaccgaatataagaaggagaaaggtttcatcctcacttcccaaaaggaggatgaaatcatgaaggtgcagaacaactcagtcatcatcaactgtgatgggttttatctcatctccctgaagggctacttctcccaggaagtcaacattagccttcattaccagaaggatgaggagcccctcttccaactgaagaaggtcaggtctgtcaactccttgatggtggcctctctgacttacaaagacaaagtctacttgaatgtgaccactgacaatacctccctggatgacttccatgtgaatggcggagaactgattcttatccatcaaaatcctggtgaattctgtgtcctttga

SEQ ID NO: 12 - DNA, 1134 bp, Artificial Sequence
  CCR7
atggacctggggaaaccaatgaaaagcgtgctggtggtggctctccttgtcattttccaggtatgcctgtgtcaagatgaggtcacggacgattacatcggagacaacaccacagtggactacactttgttcgagtctttgtgctccaagaaggacgtgcggaactttaaagcctggttcctccctatcatgtactccatcatttgtttcgtgggcctactgggcaatgggctggtcgtgttgacctatatctatttcaagaggctcaagaccatgaccgatacctacctgctcaacctggcggtggcagacatcctcttcctcctgacccttcccttctgggcctacagcgcggccaagtcctgggtcttcggtgtccacttttgcaagctcatctttgccatctacaagatgagcttcttcagtggcatgctcctacttctttgcatcagcattgaccgctacgtggccatcgtccaggctgtctcagctcaccgccaccgtgcccgcgtccttctcatcagcaagctgtcctgtgtgggcatctggatactagccacagtgctctccatcccagagctcctgtacagtgacctccagaggagcagcagtgagcaagcgatgcgatgctctctcatcacagagcatgtggaggcctttatcaccatccaggtggcccagatggtgatcggctttctggtccccctgctggccatgagcttctgttaccttgtcatcatccgcaccctgctccaggcacgcaactttgagcgcaacaaggccatcaaggtgatcatcgctgtggtcgtggtcttcatagtcttccagctgccctacaatggggtggtcctggcccagacggtggccaacttcaacatcaccagtagcacctgtgagctcagtaagcaactcaacatcgcctacgacgtcacctacagcctggcctgcgtccgctgctgcgtcaaccctttcttgtacgccttcatcggcgtcaagttccgcaacgatctcttcaagctcttcaaggacctgggctgcctcagccaggagcagctccggcagtggtcttcctgtcggcacatccggcgctcctccatgagtgtggaggccgagaccaccaccaccttctcccca

SEQ ID NO: 13 - DNA, 1131 bp, Artificial Sequence
  CXCR5
cgtacgatgaactaccctctgacactggagatggatctggagaacctcgaagacctcttttgggagctcgacagactggacaactacaacgacacctccctcgtggaaaaccatctgtgtcccgctaccgagggacctctcatggccagctttaaggccgtgtttgtgcccgtggcttattctctgatctttctgctgggcgtgatcggcaacgtgctggtgctggtcattctggagaggcacagacagacaaggagctccaccgaaaccttcctcttccatctcgccgtggccgatctgctgctggtcttcattctgcccttcgccgtggctgaaggcagcgtgggctgggtgctgggcacatttctgtgtaagaccgtgatcgctctgcacaaggtcaacttctactgctcctctctgctgctggcttgcatcgccgtggacagatatctggctattgtgcacgctgtccacgcctatagacatagaagactgctgtccatccacattacatgcggcaccatctggctcgtgggatttctgctggctctgcccgaaattctgtttgctaaggtgagccaaggccaccacaacaactctctgcctaggtgcacctttagccaagagaaccaagccgagacccacgcttggttcacatctaggttcctctatcacgtggccggctttctgctgcccatgctcgtgatgggctggtgctacgtgggcgtcgtgcacagactgagacaagcccagaggagacctcagaggcagaaggccgtgagggtggccattctggtgaccagcatcttctttctgtgctggtccccctaccacatcgtcatctttctggacacactggctagactgaaagccgtggacaacacatgcaagctcaatggatctctgcccgtggctatcaccatgtgcgagtttctgggactggctcattgctgtctgaaccccatgctgtacaccttcgctggagtcaagtttaggagcgatctgtctaggctgctgaccaaactgggctgtaccggccccgcctctctgtgccagctgtttcctagctggaggaggtcctctctgtccgagagcgaaaacgccacctctctgaccaccttctgaggatcc

SEQ ID NO: 14 - protein, 521 aa, Artificial Sequence
  Ab10
METDTLLLWVLLLWVPGSTGTGDIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVGGGGSGGGGSGGGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPRT

SEQ ID NO: 15 - protein, 493 aa, Artificial Sequence
  20
AATMETDTLLLWVLLLWVPGSTGTGQIVLSQSPAILSASPGEKVTMTCRASSSVSYIHWFQQKPGSSPKPWIYATSNLASGVPVRFSGSGSGTSYSLTISRVEAEDAATYYCQQWTSNPPTFGGGTKLEIKGGGGSGGGGSGGGGSQVQLQQPGAELVKPGASVKMSCKASGYTFTSYNMHWVKQTPGRGLEWIGAIYPGNGDTSYNQKFKGKATLTADKSSSTAYMQLSSLTSEDSAVYYCARSTYYGGDWYFNVWGAGTTVTVSAAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 518 aa, Artificial Sequence
  Ab362
METDTLLLWVLLLWVPGSTGTGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKGGGGSGGGGSGGGGSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPRT

SEQ ID NO: 17 - protein, 598 aa, Artificial Sequence
  Ab10-OX40
IGLINPGSGGTNYNEKFKGKATITADKSTSTAYMELSSLRSEDTAVYYCARVYYGNSFAYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPRTMCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 18 - protein, 704 aa, Artificial Sequence
  Ab10-OX40L
METDTLLLWVLLLWVPGSTGTGDIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVGGGGSGGGGSGGGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPRTMERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 19 - protein, 771 aa, Artificial Sequence
  Ab362-OX40
METDTLLLWVLLLWVPGSTGTGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKGGGGSGGGGSGGGGSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRMCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 20 - protein, 677 aa, Artificial Sequence
  Ab362-OX40L
METDTLLLWVLLLWVPGSTGTGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKGGGGSGGGGSGGGGSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRMERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 21 - protein, 893 aa, Artificial Sequence
  Ab10-CXCR5
METDTLLLWVLLLWVPGSTGTGDIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVGGGGSGGGGSGGGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPRTMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 22 - protein, 899 aa, Artificial Sequence
  Ab10-CCR7
METDTLLLWVLLLWVPGSTGTGDIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVGGGGSGGGGSGGGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRGSGATNFSLLKQAGDVEENPGPRTMDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 23 - protein, 276 aa, Artificial Sequence
  OX40
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 24 - protein, 183 aa, Artificial Sequence
  OX40L
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 25 - protein, 378 aa, Artificial Sequence
  CCR7
MDLGKPMKSVLVVALLVIFQVCLCQDEVTDDYIGDNTTVDYTLFESLCSKKDVRNFKAWFLPIMYSIICFVGLLGNGLVVLTYIYFKRLKTMTDTYLLNLAVADILFLLTLPFWAYSAAKSWVFGVHFCKLIFAIYKMSFFSGMLLLLCISIDRYVAIVQAVSAHRHRARVLLISKLSCVGIWILATVLSIPELLYSDLQRSSSEQAMRCSLITEHVEAFITIQVAQMVIGFLVPLLAMSFCYLVIIRTLLQARNFERNKAIKVIIAVVVVFIVFQLPYNGVVLAQTVANFNITSSTCELSKQLNIAYDVTYSLACVRCCVNPFLYAFIGVKFRNDLFKLFKDLGCLSQEQLRQWSSCRHIRRSSMSVEAETTTTFSP

SEQ ID NO: 26 - protein, 374 aa, Artificial Sequence
  CXCR5
RTMNYPLTLEMDLENLEDLFWELDRLDNYNDTSLVENHLCPATEGPLMASFKAVFVPVAYSLIFLLGVIGNVLVLVILERHRQTRSSTETFLFHLAVADLLLVFILPFAVAEGSVGWVLGTFLCKTVIALHKVNFYCSSLLLACIAVDRYLAIVHAVHAYRHRRLLSIHITCGTIWLVGFLLALPEILFAKVSQGHHNNSLPRCTFSQENQAETHAWFTSRFLYHVAGFLLPMLVMGWCYVGVVHRLRQAQRRPQRQKAVRVAILVTSIFFLCWSPYHIVIFLDTLARLKAVDNTCKLNGSLPVAITMCEFLGLAHCCLNPMLYTFAGVKFRSDLSRLLTKLGCTGPASLCQLFPSWRRSSLSESENATSLTTF

SEQ ID NO: 27 - protein, 22 aa, Artificial Sequence
  2A
GSGATNFSLLKQAGDVEENPGP

SEQ ID NO: 28 - protein, 271 aa, Artificial Sequence
  Ab10
METDTLLLWVLLLWVPGSTGTGDIVMTQSPDSLAVSLGERATISCKSSQSLLNSGNQKNYLTWYQQKPGQPPKLLIYWASTRESGVPDRFSGSGSGTDFTLTISSLQAEDVAVYYCQNDYFYPFTFGQGTKLEIKRTVGGGGSGGGGSGGGGSQVQLVQSGAEVKKPGSSVKVSCKASGYAFSNYLIEWVKQAPGQGLEWIGLINPGSGGTNYNEKFKGKATITADKSTSTAYMELSSLRSEDTAVYYCARVYYGNSFAYWGQGTLVTVSS

SEQ ID NO: 29 - protein, 268 aa, Artificial Sequence
  Ab362
METDTLLLWVLLLWVPGSTGTGDIVMTQSPSSLTVTAGEKVTMSCKSSQSLLNSGNQKNYLTWYQQKPGQPPKLLIYWASTRESGVPDRFTGSGSGTDFTLTISSVQAEDLAVYYCQNDYSYPFTFGSGTKLEIKGGGGSGGGGSGGGGSQVQLQQPGAELVRPGASVKLSCKASGYTFTSYWINWVKQRPGQGLEWIGNIYPSDSYTNYNQKFKDKATLTVDKSSSTAYMQLSSPTSEDSAVYYCTRSWRGNSFDYWGQGTTLTVSS

SEQ ID NO: 30 - protein, 45 aa, Artificial Sequence
  hinge region
TTTPAPRPPTPAPTIASQPLSLRPEACRPAAGGAVHTRGLDFACD

SEQ ID NO: 31 - protein, 24 aa, Artificial Sequence
  transmembrane domain
IYIWAPLAGTCGVLLLSLVITLYC

SEQ ID NO: 32 - protein, 42 aa, Artificial Sequence
  co-stimulatory domain
KRGRKKLLYIFKQPFMRPVQTTQEEDGCSCRFPEEEEGGCEL

SEQ ID NO: 33 - protein, 112 aa, Artificial Sequence
  intracellular signaling transduction domain
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR